(19) United States
(12) Patent Application Publication
(10) Pub. No.: US 2022/0226508 A1
(43) Pub. Date: Jul. 21, 2022

(54) METHODS AND COMPOSITIONS FOR TREATING EPILEPSY

(71) Applicant: THE REGENTS OF THE UNIVERSITY OF CALIFORNIA, Oakland, CA (US)

(72) Inventors: Scott C. BARABAN, Novato, CA (US); Aliesha L. GRIFFIN, San Francisco, CA (US); Adam R. RENSLO, Oakland, CA (US); Priyadarshini JAISHANKAR, San Francisco, CA (US)

(21) Appl. No.: 17/595,823

(22) PCT Filed: May 28, 2020
(86) PCT No.: PCT/US2020/034981

§ 371 Date: Nov. 24, 2021

Related U.S. Application Data
(60) Provisional application No. 62/853,971, filed on May 29, 2019.

(51) Int. Cl. A61K 49/00

(52) U.S. Cl. CPC A61K 49/0008

(57) ABSTRACT

Provided, inter alia, are methods and compositions for treating epilepsy. In one aspect, provided herein is a method of selecting a compound for treating epilepsy, said method includes, contacting a test compound with 5-hydroxytryptamine-2B receptor (5-HT2B), and measuring the 5-HT2B agonistic activity of the test compound. In another aspect, provided herein is a method of treating an epilepsy in a subject in need thereof. The method includes administering to said subject an effective amount of a 5-HT2B specific receptor agonist.

### CROSS-REFERENCES TO RELATED APPLICATIONS

This application claims priority to U.S. Provisional Application No. 62/853,971 filed May 29, 2019, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with Government support under grant number R01 NS096976 awarded by the National Institutes of Health. The Government has certain rights in the invention.

### BACKGROUND

Dravet syndrome (DS) is a catastrophic pediatric epilepsy with severe intellectual disability, impaired social development and persistent drug-resistant seizures. One of its primary causes is mutations in Na_{V}1.1 (SCN1A), a voltage-gated sodium channel. Seizures experienced by those with DS and other epilepsy disorders are inadequately managed using available antiepileptic drugs (AEDs) and children with DS are poor candidates for neurosurgical resection. Thus there is a need in the art for epilepsy treatment options, especially those for DS and related catastrophic pediatric epilepsies. Provided herein are solutions to these problems and other problems in the art.

### BRIEF SUMMARY

Provided herein, inter alia, are methods and compositions for treating epilepsy. In one aspect, provided herein is a method of selecting a compound for treating epilepsy, said method includes, contacting a test compound with 5-hydroxytryptamine-2B receptor (5-HT_{2B}), and measuring the 5-HT_{2B} agonistic activity of the test compound.

In another aspect, provided herein is a method of selecting a compound for treating epilepsy, said method includes contacting a test compound with 5-HT_{2B} receptor, and measuring the 5-HT_{2B} agonistic activity of the test compound. The method further includes administering the test compound to an epileptic animal model and measuring the behavioral activity in said epileptic animal model.

In another aspect, provided herein is a method of treating an epilepsy in a subject in need thereof. The method includes administering to said subject an effective amount of a 5-HT_{2B} specific receptor agonist.

### BRIEF DESCRIPTION OF THE DRAWINGS

FIG. 1 (A-B): Phenotypic screening of elnizole analogs. Twenty-eight clemizole analogs were screened for efficacy in suppressing the high-velocity seizure-like swim behavior observed in scn1lab mutant zebrafish. Plots show the change in mean swim velocity of 5 dpf larvae screened at (FIG. 1A) 100 μM, or (FIG. 1B) 250 μM. Threshold for inhibition of seizure activity (positive hits—labeled data points) was determined as a reduction in mean swim velocity of ≥40% (dashed line). The data points below the dashed line, other than the labeled points, represent compounds that were classified as toxic after 90-min exposure. The heat map shows the % change in velocity for the six individual larva from the first trial (1-6). Mean velocity change from six individual fish is shown for trial 1 and 2. Clemizole analog 25 (*) failed to go into solution at 250 μM so it was not considered for further testing.

FIG. 2 (A-I): Evaluation of clemizole analogs that reduce seizure-like swim behavior in scn1lab mutant zebrafish. Clemizole analogs identified as positive from the in vivo screen were freshly synthesized and retested for efficacy in suppressing the seizure-like behavior of 5 dpf scn1Lab mutant zebrafish. Graphs show the change in mean velocity over four concentrations of (FIG. 2A) compound 4, (FIG. 2B) compound 6 and (FIG. 2C) compound 20. Each bar represents the mean change in velocity±SEM from three independent experiments (six individual larva per experiment). Toxicity is indicated by dashed bars. The threshold for a decrease in velocity is ≥40% (dashed line). Locomotion of larvae was recorded for 10 min after an exposure of 30 min (black bars) and 90 min (gray bars). A representative raw 10 min tracking plot is shown for a single experiment of six individual scn1Lab zebrafish. The chemical structure for each clemizole analog is shown (FIG. 2D-2F). In vitro radioligand binding analyses of (FIG. 2G) compound 4, (FIG. 2H) compound 6 and (FIG. 2I) compound 20 revealed specificity for 5-HT_{2B}R over 5-HT2R subtypes. Compound SB206553 was used as a positive control for 5-HT_{2B}R binding.

FIG. 3 (A-H): Dose response evaluation of 5HT_{2B}R agonists in scn1lab mutant zebrafish. 5HT_{2B}R agonists were tested for efficacy in reducing the high-speed seizure-like behavior in 5 dpf scn1lab mutant zebrafish. Graphs show the change in mean velocity over three concentrations of (FIG. 3A) methylergonovine, (FIG. 3B) 6-APB, (FIG. 3C) norfenfluamine, (FIG. 3D) BW-723C86, (FIG. 3E) RO-60-0175, (FIG. 3F) TL-99, (FIG. 3G) m-CPP, and (FIG. 3H) CP-809,101. Larvae locomotion was recorded for 10 min after an exposure of 30 min (black bars) and 90 min (gray bars). Each bar represents the mean change in velocity±SEM from three independent experiments (six individual larva per experiment). The threshold for a decrease in velocity is ≥40% (dashed line). Representative tracking plots of a 10 min recording are shown for six individual 5 dpf scn1lab zebrafish at baseline, and following 30 min and 90 min exposure of 100 μM of each compound.

FIG. 4 (A-C): Electrophysiological assay to identify drugs that rescue the scn1lab mutant epilepsy phenotype. (FIG. 4A) Electrophysiology recording were obtained with an electrode placed in the forebrain of 5 dpf agar-immobilized scn1lab larvae that had previously showed suppressed seizure-like behavior in the locomotion assay. (FIG. 4B) Bar graphs show the frequency of epileptiform events in a 10 min recording epoch for scn1lab larvae exposed to clemizole analogs 4 (n=15), 6 (n=12), 20 (n=9), 6-APB (n=6), norfenfluramine (NorFA) (n=8), or methylergonovine (MeERGO) (n=7), or scn1lab mutants (n=15). The graph shows mean±SEM and individual data points are shown. (FIG. 4C) Representative field electrode recording epochs (10 min) are shown for clemizole analogs 4, 6, 20, methylergonovine (MeERGO) and 6-APB. These compounds showed significant changes in the frequency of events compared to untreated scn1lab mutant zebrafish (top graph).

FIG. 5 (A-D): Behavioral screening of resynthesized clemizole analogs 5 and 14. Clemizole analogs (FIG. 5A) 5 and (FIG. 5B) 14 were identified as having specific binding for 5HT_{2B}R. Subsequently, they were independently synthesized and tested. Behavioral testing confirmed the previous screening results and showed no significant effect on the high-speed seizure-like behavior in 5 dpf scn1lab mutant zebrafish. Graphs show the change in mean velocity of six fish treated with each clemalog (FIG. 5C and FIG. 5D). The threshold for a decrease in velocity is ≥40% (dashed line). Locomotion of larvae was recorded for 10 min after an exposure of 30 min (black bars) and 90 min (gray bars). The raw 10 min tracking plot is shown for the baseline, 30 min and 90 min exposure of 100 μM.

FIG. 6 (A-D): Dose response evaluation of 5HT_{2B}R agonists in scn1lab mutant zebrafish. 5HT_{2B}R agonists were tested for efficacy in reducing the high-speed seizure-like behavior in 5 dpf scn1lab mutant zebrafish. Graphs show the change in mean velocity over three concentrations of (FIG. 6A) cabergoline, (FIG. 6B) bromocriptine, (FIG. 6C) apomorphine, and (FIG. 6D) piribedil. Larvae locomotion was recorded for 10 min after an exposure of 30 min (black bars) and 90 min (gray bars). Each bar represents the mean change in velocity±SEM from three independent experiments (six individual larva per experiment). The threshold for a decrease in velocity is ≥40% (dashed line). Toxicity is indicated by dashed bars. Representative tracking plots of a 10 min recording are shown for six individual 5 dpf scn1lab zebrafish at baseline and following 30 min and 90 min exposure of 100 μM of each compound.

### DETAILED DESCRIPTION

### I. Definitions

“5HT Receptor” “5-HT Receptor” or “5-Hydroxytryptamine Receptor” refers to a group of G protein-coupled receptors (GPCRs) and ligand-gated ion channels (LGICs) that are found in the central (CNS) and peripheral nervous systems (PNS) and belong generally to the group of serotonin receptors. 5HT receptors may be divided into seven receptor families, including: 5HT₁ (e.g., Gᵢ/G₀-protein coupled receptors), 5HT₂ (e.g., G_{q}/G₁₁-protein coupled receptors), 5HT₃ (e.g., ligand-gated Na⁺ and K⁺ cation channel), 5HT₄ (e.g., Gₛ-protein coupled receptors), 5HT₅ (e.g., Gᵢ/G₀-protein coupled receptors), 5HT₆ (e.g., Gₛ-protein coupled receptors), and 5HT₇ (e.g., Gₛ-protein coupled receptors). Additionally, the seven families of 5HT receptors may be further subdivided into a number of sub-families. For example, the 5HT₁ family also includes the following sub-families: 5HT_{1A} (e.g., which are known to function in blood vessels and the CNS and may be involved in addiction, aggression, anxiety, appetite, autoreceptor, blood pressure, cardiovascular function, emesis, heart rate, impulsivity, memory, mood, nausea, nociception, penile erection, pupil dilation, respiration, sexual behavior, sleep, sociability, thermoregulation, and vasoconstriction), 5HT_{1B} (e.g., which are known to function in blood vessels and the CNS and may be involved in addiction, aggression, anxiety, auto receptor, learning, locomotion, memory, mood, penile erection, sexual behavior, and vasoconstriction), 5HT_{1D} (e.g., which are known to function in blood vessels and the CNS and may be involved in anxiety, autoreceptor, locomotion, and vasoconstriction), 5HT_{1E} (e.g., which are known to function in the CNS and may be involved in migraines). As another example, the 5HT₂ family may be divided into the following sub-families: 5HT_{2A} (e.g., which are known to function in blood vessels, CNS, gastrointestinal tract, platelets, PNS, and smooth muscle, and may be involved in addiction, anxiety, appetite, cognition, imagination, learning, memory, mood, perception, sexual behavior, sleep, thermoregulation, and vasoconstriction), 5HT_{2B} (e.g., which are known to function in blood vessels, CNS, gastrointestinal tract, platelets, PNS, and smooth muscle, and may be involved in anxiety, appetite, cardiovascular function, gastrointestinal motility, sleep, and vasoconstriction), and 5HT_{2C} (e.g., which are known to function in blood vessels, CNS, gastrointestinal tract, platelets, PNS, and smooth muscle, and may be involved in addiction, anxiety, appetite, gastrointestinal motility, locomotion, mood, penile erection, sexual behavior, sleep, thermoregulation, and vasoconstriction). Additionally, the 5HT₅ family may be further divided into the following sub-families: 5HT_{5A} (e.g., which may function in the CNS, and play a role in locomotion and sleep, as well as function as an autoreceptor) and 5HT₅B (e.g., which may function in rodents and appears to be a pseudogene in humans).

“5HT Receptor agonist” or “5-HT Receptor agonist” refers to any agent that activates a 5HT receptor relative to the absence of the 5HT Receptor agonist or in a manner similar to serotonin. Exemplary 5HT receptor agonists include, but are not limited to, any one or more of the following: ACP-104, ACP-106, AR-116081, AR-116082, ATHX-105, belladonna in combination with ergotamine tartrate, BW 723C86, Cisapride, Ciza-MPS, Cizap, Cizap-Mps, CSC-500 Series, DOI or salt thereof, Ergotamine Tartrate and Caffeine, Esorid MPS, flibanserin, Ikaran L. P., Manotac Plus, Migril, Mirtazapina Rimafar, mirtazapine, Naratriptan, nelotanserin, norfenfluramine, Normagut Tab, nefazodone hydrochloride, OSU-6162, Pridofin, Sensiflu, PRX-00933, RP-5063, Small Molecule to Agonize 5-HT_{2A} for Inflammatory Diseases, Small Molecules to Agonize 5-HT_{2C} for Schizophrenia and Obesity, Small Molecules to Agonize 5-HT_{2C} Receptor for Obesity, Small Molecules to Target 5-HT_{2C} and 5-HT₆ Receptor for Schizophrenia, Small Molecules to Modulate 5HT₂ for CNS and Metabolic Disorders, TGBA-01AD, trazodone hydrochloride, temanogrel hydrochloride, vabicaserin hydrochloride, Virdex, VR-1065, ziprasidone hydrochloride, and Ziprasidon-Sigillata. It is further contemplated within the scope of the disclosure that in some embodiments, a 5HT Receptor agonist does not include one or more or all of the following: acetazolamide, benzodiazepine (diazepam; clobazam), cannabadiol, carbamazepine, clemizole, ethosuximide, felbamate, fenfluramine, fluoxetine, gabapentin, ganaxolone, lacosamide, lamotrigine, levetiracetam, nitrazepam, oxcarbazepine, perampenel, phenytoin, phenobarbital, piracetam, potassium bromide, pregabalin, primidone, retigabine, rufinamide, stiripentol, tiagabine, topiramate, valproic acid, verapamil, vigabatrin, and zonisamide.

“5-HT_{2B} specific receptor agonist” refers to any agent that is capable of agonizing 5-HT_{2B} receptor to a greater degree than 5-HT_{2A} receptor. In embodiments, the 5-HT_{2B} specific receptor agonist is an agent that has low binding activity to 5-HT_{2A} receptor or 5-HT_{2C} receptor or does not demonstrate measurable 5-HT_{2A} receptor or 5-HT_{2C} receptor binding activity. In embodiments, the 5-HT_{2B} specific receptor agonist is an agent whose agonistic activity is 10, 100, 1000, 10000, or 100000 times greater for 5-HT_{2B} receptor relative to 5-HT_{2A} receptor. The times greater activity may be a value selected from the range 10 to 100, 100 to 1000, 1000 to 10000, or 10000 to 100000. In embodiments, the 5-HT_{2B} specific receptor agonist is an agent, wherein said agent binds to HT_{2B} receptor with a Kd of less than 100 nM, 10 nM, 1 nM, 500 pM, or 100 pM. The Kd may be a specific value in a range from 100 pM to 500 pM, 500 pM to 1 nM, 1 nM to 10 nM, or 10 nM to 100 nM. The specific value may be selected from any 1 pM increment in the range. The Kd may be selected from a sub-range within any of the aforementioned Kd ranges. The low endpoint of a sub-range may be the low end of the range or any value selected from 1 pM increments above the low end of the range up to 1 pM less that the high end of the range. The high endpoint of a sub-range may be the high end of the range or any value selected from 1 pM below the high end of the range to 1 pM greater than the low end of the range.

“Analog” or “analogue” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound.

“Clemizole” refers to a compound having formula:

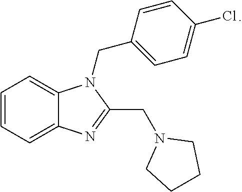 [SMILES: Clc1ccc(Cn2c(CN3CCCC3)nc3ccccc32)cc1]

In embodiments, clemizole composition may include pharmaceutically acceptable salts of clemizole as described herein (e.g. a “clemizole salt”). Exemplary, clemizole salts include but are not limited to clemizole-HCl, clemizolpenicillin, clemizole-sulfate, or clemizole-undecylate. In embodiments, the clemizole composition does not include clemizole salts having a pharmaceutically active ingredient. In embodiments, if clemizole composition includes a salt, clemizole salt is not clemizole topiramate. In embodiments, clemizole composition may include formulations of clemizole.

A “clemizole analog” as set forth herein refers to compounds of similar structure. Such compounds include, for example, those compounds set forth in PCT/US2008/076804, and U.S. Pat. No. 4,011,322, which are herein incorporated by reference in their entirety. Further exemplary clemizole analogs are set forth, for example in: US 2012/0232062; PCT Pub. Nos. 2009/038248; US 2010/107739; US 2010/107742, WO 2002/089731. WO 2005/032329, WO 2009/039248, WO 2010/039195, WO 2010/107739, and WO 2010/107742, each of which is incorporated herein by reference in their entirety. Clemizole analogs described herein (including the compounds described in the references above) may be substituted (i.e. modified) at the 1 or 2 position as set forth below in formula (I) (boxes Y and Z). Clemizole analogs may be substituted (i.e. modified) at 4, 5, 6, or 7 positions as indicated by box X in formula (I).

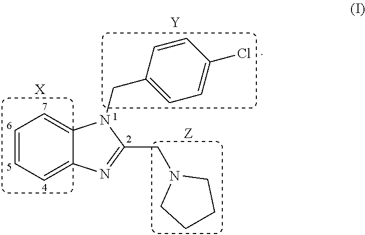

“Flibanserin” refers to a compound having the following formula (II):

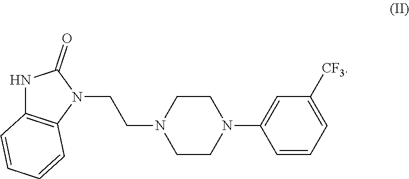 [SMILES: O=c1[nH]c2ccccc2n1CCN1CCN(c2cccc(C(F)(F)F)c2)CC1]

In embodiments, flibanserin composition may include pharmaceutically acceptable salts of flibanserin (e.g. a “flibanserin salt”). In embodiments, flibanserin composition may include formulations of flibanserin.

“Norfenfluramine” refers to a compound having the following formula (III):

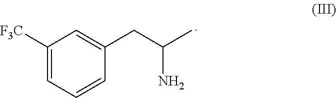

In embodiments, norfenfluramine composition may include pharmaceutically acceptable salts of norfenfluramine (e.g. a “norfenfluramine salt”). In embodiments, norfenfluramine composition may include formulations of norfenfluramine.

“DOI” refers to 2,5-dimethoxy-4-Iodoamphetamine. In embodiments, DOI composition may include pharmaceutically acceptable salts of DOI. Exemplary, DOI salts include but are not limited to 2,5-dimethoxy-4-Iodoamphetamine monohydrochloride (DOI HCl) having the following formula (IV):

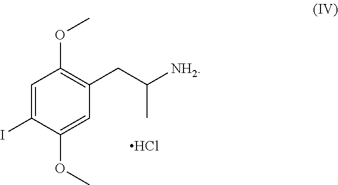

In embodiments, DOI composition may include formulations of DOI.

“BW 723C86” refers to a tryptamine derivative drug that acts as a 5HT_{2B} receptor agonist and has the following formula (V):

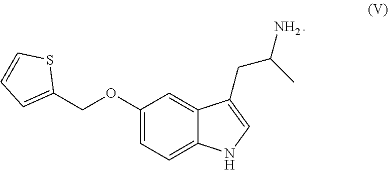 [SMILES: CC(N)Cc1c[nH]c2ccc(OCc3cccs3)cc12]

In embodiments, BW 723C86 composition may include pharmaceutically acceptable salts of BW 723C86 (e.g. a “BW 723C86 salt”). In embodiments, BW 723C85 composition may include formulations of BW 723C85.

The term “pharmaceutically acceptable salts” is meant to include salts of the active compounds that are prepared with relatively nontoxic acids or bases, depending on the particular substituents found on the compounds described herein. When the 5-HT_{2B} agonist contains relatively acidic functionalities, base addition salts can be obtained by contacting the neutral form of such compounds with a sufficient amount of the desired base, either neat or in an inert solvent. Examples of pharmaceutically acceptable base addition salts include sodium, potassium, calcium, ammonium, organic amino, or magnesium salt, or a similar salt. When the 5-HT_{2B} agonist contains relatively basic functionalities, acid addition salts can be obtained by contacting the neutral form of such compounds with a sufficient amount of the desired acid, either neat or in an inert solvent. Examples of pharmaceutically acceptable acid addition salts include those derived from inorganic acids selected from hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids selected from acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids (e.g., but not limited to, arginate and the like), and salts of organic acids (e.g., but not limited to, glucuronic or galactunoric acids and the like) (see, for example, Berge et al., “Pharmaceutical Salts”, Journal of Pharmaceutical Science, 1977, 66, 1-19). The 5-HT_{2B} agonist may contain both basic and acidic functionalities that allow conversion into either base or acid addition salts.

The 5-HT_{2B} agonist may exist as a salt, such as with pharmaceutically acceptable acids. Non-limiting examples of such salts include hydrochlorides, hydrobromides, phosphates, sulfates, methanesulfonates, nitrates, maleates, acetates, citrates, fumarates, proprionates, tartrates (e.g., but not limited to (+)-tartrates, (−)-tartrates, or mixtures thereof including racemic mixtures), succinates, benzoates, and salts with amino acids such as glutamic acid, and quaternary ammonium salts (e.g., but not limited to methyl iodide, ethyl iodide, and the like). These salts may be prepared by methods known to those skilled in the art. The neutral forms of the 5-HT_{2B} agonist are preferably regenerated by contacting the salt with a base or acid and isolating the parent compound in the conventional manner. The parent form of the compound may differ from the various salt forms in certain physical properties, such as solubility in polar solvents.

In addition to salt forms, the 5-HT_{2B} agonist may be provided in a prodrug form. Prodrugs of are those compounds that undergo chemical changes under physiological conditions to provide the 5-HT_{2B} agonist. Prodrugs of the 5-HT_{2B} agonist may be converted in vivo after administration. Additionally, prodrugs of the 5-HT_{2B} agonist may be converted to active compounds by chemical or biochemical methods in an ex vivo environment (e.g., but not limited to, when contacted with a suitable enzyme or chemical reagent).

The 5-HT_{2B} agonist may exist in unsolvated forms as well as solvated forms, including hydrated forms. In general, the solvated forms are equivalent to unsolvated forms and are encompassed within the scope of the present invention. The 5-HT_{2B} agonist may exist in multiple crystalline or amorphous forms. In general, all physical forms are equivalent for the uses contemplated by the present invention and are intended to be within the scope of the present invention.

An “effective amount” is an amount sufficient for the 5-HT_{2B} agonist (including pharmaceutically acceptable salts thereof) to accomplish a stated purpose relative to the absence of the 5-HT_{2B} agonist (including pharmaceutically acceptable salts thereof) (e.g. achieve the effect for which it is administered, treat a disease, reduce protein/enzyme activity, increase protein/enzyme activity, reduce a signaling pathway, or reduce one or more symptoms of a disease or condition). An example of an “effective amount” is an amount of the 5-HT_{2B} agonist (including pharmaceutically acceptable salts thereof) which is sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s) (e.g. seizures). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms (e.g. seizures). The full prophylactic effect does not necessarily occur by administration of one dose, and may occur only after administration of a series of doses. Thus, a prophylactically effective amount may be administered in one or more administrations. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The therapeutically effective amount of the 5-HT_{2B} agonist (including pharmaceutically acceptable salts thereof) can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

Dosages may be varied depending upon the requirements of the patient and the compound being employed. The dose administered to a patient, in the context of the present invention should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is effective to treat the clinical symptoms demonstrated by the particular patient. This planning should involve the careful choice of active compound by considering factors such as compound potency, relative bioavailability, patient body weight, presence and severity of adverse side effects, preferred mode of administration and the toxicity profile of the selected agent.

“Control” or “control experiment” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a 5-HT_{2B} agonist (including pharmaceutically acceptable salts thereof).

A “test compound” as used herein refers to an experimental compound used in a screening process to identify activity, non-activity, or other modulation of a particularized biological target or pathway, for example a 5-HT_{2B} receptor.

The term “modulation”, “modulate”, or “modulator” are used in accordance with their plain ordinary meaning and refer to the act of changing or varying one or more properties. “Modulator” refers to a composition that increases or decreases the level of a target molecule or the function of a target molecule or the physical state of the target of the molecule. “Modulation” refers to the process of changing or varying one or more properties. For example, as applied to the effects of a modulator on a biological target, to modulate means to change by increasing or decreasing a property or function of the biological target or the amount of the biological target (e.g. a 5-HT receptor).

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In some embodiments inhibition refers to reduction of a disease or symptoms of disease. In some embodiments, inhibition refers to a reduction in the activity of a particular protein or nucleic acid target. Thus, inhibition includes, at least in part, partially or totally blocking stimulation, decreasing, preventing, or delaying activation, or inactivating, desensitizing, or down-regulating signal transduction or protein/enzymatic activity or the amount of a protein. Inhibition as used herein may refer to inhibition of a voltage-gated sodium channel.

The term “activation” or “activating” and the like refer to protein-compound interactions that positively affect (e.g. increase) the activity or function of the protein relative to the activity or function of the protein in absence of the activator compound. Activation may refer to enhanced activity of a particular protein target. Activation may refer to restoration of loss-of-function of a mutated protein target. Activation as used herein may refer to activation of one or more 5-HT receptors.

“Contacting” is used in accordance with its plain ordinary meaning and refers to the process of allowing at least two distinct species (e.g., but not limited to chemical compounds, biomolecules, or cells) to become sufficiently proximal to react, interact or physically touch. It should be appreciated; however, the resulting reaction product may be produced directly from a reaction between the added reagents or from an intermediate from one or more of the added reagents that can be produced in the reaction mixture.

The term “contacting” may include allowing two species to react, interact, or physically touch, wherein the two species may be a compound as described herein and a protein or enzyme. In some embodiments contacting includes allowing a compound described herein to interact with a receptor, e.g. a 5-HT_{2B} receptor.

The term “associated” or “associated with” in the context of a substance or substance activity or function associated with a disease means that the disease is caused by (in whole or in part), or a symptom of the disease is caused by (in whole or in part) the substance or substance activity or function.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, zebrafish, and other non-mammalian animals. A patient may be human.

“Disease” or “condition” refer to a state of being or health status of a patient or subject capable of being treated with the compounds or methods provided herein.

The terms “epileptic disorder,” “epilepsy disorder,” “seizure disorder,” or “epilepsy” herein refer to a spectrum of chronic neurological disorders most often characterized by the presence of unprovoked seizures. See e.g. Noebels et. al., Jasper's Basic Mechanisms of the Epilepsies, 4th edition, Bethesda (Md.): National Center for Biotechnology Information (US); 2012. Epilepsy as used herein, may refer to injury to the brain (e.g. from trauma, stroke, or cancer) or genetic mutation. The symptoms of epilepsy disorders may result from abnormal electrochemical signaling between neurons in the brain. Patients experiencing two or more unprovoked seizures may be considered to have epilepsy.

Types of epilepsy include, for example, benign Rolandic epilepsy, frontal lobe epilepsy, infantile spasms, juvenile myoclonic epilepsy (JME), juvenile absence epilepsy, childhood absence epilepsy (e.g. pyknolepsy), febrile seizures, progressive myoclonus epilepsy of Lafora, Lennox-Gastaut syndrome, Landau-Kleffner syndrome, Dravet syndrome (DS), Generalized Epilepsy with Febrile Seizures (GEFS+), Severe Myoclonic Epilepsy of Infancy (SMEI), Benign Neonatal Familial Convulsions (BFNC), West Syndrome, Ohtahara Syndrome, early myoclonic encephalopathies, migrating partial epilepsy, infantile epileptic encephalopathies, Tuberous Sclerosis Complex (TSC), focal cortical dysplasia, Type I Lissencephaly, Miller-Dieker Syndrome, Angelman's syndrome, Fragile X syndrome, epilepsy in autism spectrum disorders, subcortical band heterotopia, Walker-Warburg syndrome, Alzheimer's disease, posttraumatic epilepsy, progressive myoclonus epilepsies, reflex epilepsy, Rasmussen's syndrome, temporal lobe epilepsy, limbic epilepsy, status epilepticus, abdominal epilepsy, massive bilateral myoclonus, catamenial epilepsy, Jacksonian seizure disorder, Unverricht-Lundborg disease, or photosensitive epilepsy.

“Pharmaceutically acceptable excipient” and “pharmaceutically acceptable carrier” or “carrier moiety” refer to a substance that aids the administration of the 5-HT_{2B} agonist (including pharmaceutically acceptable salts thereof) to and absorption by a subject and can be included in the compositions without causing a significant adverse toxicological effect on the subject. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates (e.g., but not limited to, lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds herein). One of skill in the art will recognize that other pharmaceutically acceptable excipients are useful in the present invention.

The term “preparation” is intended to include the formulation of the 5-HT_{2B} agonist (including pharmaceutically acceptable salts thereof) with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The 5-HT_{2B} agonists (including pharmaceutically acceptable salts thereof) and pharmaceutical compositions thereof may be delivered transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The 5-HT_{2B} agonists (including pharmaceutically acceptable salts thereof) may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The 5-HT_{2B} agonists (including pharmaceutically acceptable salts thereof) may also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). The formulations of the compositions of the 5-HT_{2B} agonists (including pharmaceutically acceptable salts thereof) may be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the 5-HT_{2B} agonists (including pharmaceutically acceptable salts thereof) into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions may also be delivered as nanoparticles.

By “co-administer” it is meant that a composition described herein is administered at the same time, just prior to, or just after the administration of one or more additional therapies. The 5-HT_{2B} agonist (including pharmaceutically acceptable salts thereof) may be administered alone or can be co-administered to the patient. Co-administration is meant to include simultaneous or sequential administration of the compounds individually or in combination (more than one compound). Thus, the preparations may also be combined, when desired, with other active substances (e.g. to reduce metabolic degradation). The 5-HT_{2B} agonists (including pharmaceutically acceptable salts thereof) may be delivered transdermally, by a topical route, or formulated as applicator sticks, solutions, suspensions, emulsions, gels, creams, ointments, pastes, jellies, paints, powders, and aerosols.

The terms “add on therapy,” “add-on therapy,” “adjunct therapy,” and “adjunctive therapy” are used interchangeably herein and refer to combining the 5-HT_{2B} agonist, or a pharmaceutically acceptable salt thereof with another anticonvulsant to treat epilepsy.

An “anti-seizure drug”, “anti-epilepsy drug”, “AED” or “anticonvulsant” are used interchangeably herein and according to their common and ordinary meaning and include compositions for reducing or eliminating seizures. Anticonvulsants include, but are not limited to acetazolamide, benzodiazepine, cannabadiols, carbamazepine, clobazam, clonazepam, eslicarbazepine acetate, ethosuximide, ethotoin, felbamate, fenfluramine, fosphenytoin, gabapentin, ganaxolone, huperzine A, lacosamide, lamotrigine, levetiracetam, nitrazepam, oxcarbazepine, perampanel, piracetam, phenobarbital, phenytoin, potassium bromide, pregabalin, primidone, retigabine, rufinamide, sodium valproate, stiripentol, tiagabine, topiramate, vigabatrin, or zonisamide.

As used herein, the term “resistant” refers to a reduction in the effectiveness of an agent or drug. For example, a subject that is “resistant” to treatment with a serotonin reuptake inhibitor (such as, e.g. fenfluramine) displays a reduced response relative to the response observed when the subject was first treated with the serotonin reuptake inhibitor.

### I. Methods of Treatment

Provided herein, inter alia, are methods and compositions for treating epilepsy. In one aspect, provided herein is a method of selecting a compound for treating epilepsy, said method includes, contacting a test compound with 5-hydroxytryptamine-2B receptor (5-HT_{2B}), and measuring the 5-HT_{2B} agonistic activity of the test compound. The method may include selecting as the compound a test compound that demonstrates 5-HT_{2B} agonistic activity. The compound selected may have a low binding activity to 5-HT_{2A} receptor or 5-HT_{2C} receptor, or the compound selected may not demonstrate measurable 5-HT_{2A} receptor or 5-HT_{2C} receptor binding activity. A method of treating epilepsy may comprise administering the compound selected to a patient or subject in need thereof.

In another aspect, provided herein is a method of selecting a compound for treating epilepsy, said method includes contacting a test compound with 5-HT_{2B} receptor, and measuring the 5-HT_{2B} agonistic activity of the test compound. The method further includes administering the test compound to an epileptic animal model and measuring the behavioral activity in said epileptic animal model. The animal model may be scn1lab mutant zebrafish. The agonistic activity of the test compound may be measured by one or more of: convulsive high-velocity swim behavior or spontaneous electrographic seizures in the scn1lab mutant zebrafish, or 5-HT_{2B} binding activity of the test compound.

In another aspect, provided herein is a method of treating an epilepsy in a subject in need thereof. The method includes administering to said subject an effective amount of a 5-HT_{2B} specific receptor agonist. The agonistic activity of said 5-HT_{2B} specific receptor agonist may be 10, 100, 1000, 10000 or 100000 times greater relative to its 5-HT_{2A} receptor agonistic activity. The times greater activity may be a value selected from the range 10 to 100, 100 to 1000, 1000 to 10000, or 10000 to 100000. The 5-HT_{2B} specific receptor agonist may bind to 5-HT_{2B} receptor with a Kd of less than 100 nM, 10 nM, 1 nM, 500 pM or 100 pM. The Kd may be a specific value in a range from 100 pM to 500 pM, 500 pM to 1 nM, 1 nM to 10 nM, or 10 nM to 100 nM. The specific value may be selected from any 1 pM increment in the selected range. The Kd may be selected from a sub-range within any of the aforementioned Kd ranges. The low endpoint of a sub-range may be the low end of the range or any value selected from 1 pM increments above the low end of the range up to 1 pM less that the high end of the range. The high endpoint of a sub-range may be the high end of the range or any value selected from 1 pM below the high end of the range to 1 pM greater than the low end of the range. The 5-HT_{2B} specific receptor agonist may be a compound selected by conducting a method of selecting a compound for treating epilepsy herein.

The epilepsy may be benign Rolandic epilepsy, frontal lobe epilepsy, infantile spasms, juvenile myoclonic epilepsy (JME), juvenile absence epilepsy, childhood absence epilepsy (e.g. pyknolepsy), febrile seizures, progressive myoclonus epilepsy of Lafora, Lennox-Gastaut syndrome, Landau-Kleffner syndrome, Dravet syndrome, Generalized Epilepsy with Febrile Seizures (GEFS+), Severe Myoclonic Epilepsy of Infancy (SMEI), Benign Neonatal Familial Convulsions (BFNC), West Syndrome, Ohtahara Syndrome, early myoclonic encephalopathies, migrating partial epilepsy, infantile epileptic encephalopathies, Tuberous Sclerosis Complex (TSC), focal cortical dysplasia, Type I Lissencephaly, Miller-Dieker Syndrome, Angelman's syndrome, Fragile X syndrome, epilepsy in autism spectrum disorders, subcortical band heterotopia, Walker-Warburg syndrome, Alzheimer's disease, posttraumatic epilepsy, progressive myoclonus epilepsies, reflex epilepsy, Rasmussen's syndrome, temporal lobe epilepsy, limbic epilepsy, status epilepticus, abdominal epilepsy, massive bilateral myoclonus, catamenial epilepsy, Jacksonian seizure disorder, Unverricht-Lundborg disease, or photosensitive epilepsy. The epilepsy may include generalized seizures or partial (i.e. focal) seizures. The epilepsy may be Dravet Syndrome.

The epilepsy may be a result of a neurological disease or injury such as, for example, encephalitis, cerebritis, abscess, stroke, tumor, trauma, genetic, tuberous sclerosis, cerebral dysgenesis, or hypoxic-ischemic encephalophathy. The epilepsy may be associated with a neurodegenerative disease such as, for example, Alzheimer's disease or Parkinson's Disease. The epilepsy may be associated with autism. The epilepsy may be associated with a single gene mutation. The epilepsy disease may be associated with compulsive behaviors or electrographic seizures.

The administration of the test compound may inhibit compulsive behaviors or electrographic seizures. The inhibition may be measured by measuring a behavioral activity in an epileptic animal model.

The administration of the test compound may reduce the incidence (e.g. number of occurrences) of unprovoked seizures in the epileptic animal model compared to the absence of a test compound. Thus, an epileptic animal model's response to the administration of the test compound, may be monitored progressively compared to a time before the administration of compounds described herein (e.g. a control or control time).

The administration of the test compound may reduce or prevent myoclonus seizures or status epilepticus in the epileptic animal model compared to the absence of a test compound. The administration of the test compound may reduce or prevent myoclonus seizures in the epileptic animal model compared to the absence of a test compound. The administration of the test compound may reduce or prevent status epilepticus in the epileptic animal model compared to the absence of a test compound. An epileptic animal model's response to the administration of the test compound, may be monitored progressively compared to a time before the administration of compounds described herein (e.g. a control or control time).

The epileptic animal model may be a Dravet Syndrome (DS) animal model. The DS animal model may be a zebrafish (Danio rerio). The zebrafish (Danio rerio) may be a scn1lab mutant or a scn1laa mutant. The zebrafish (Danio rerio) may be a scn1lab mutant. The zebrafish (Danio rerio) may be a scn1laa mutant.

The DS animal model may be a zebrafish (Danio rerio) that is resistant to anti-epilepsy drugs (AEDs). The zebrafish (Danio rerio) may be a scn1lab mutant or a scn1laa mutant that is resistant to AEDs. The zebrafish (Danio rerio) may be a scn1lab mutant that is resistant to AEDs. The zebrafish (Danio rerio) may be a scn1laa mutant that is resistant to AEDs.

The AED may be acetazolamide, benzodiazepine, cannabadiols, carbamazepine, clobazam, clonazepam, eslicarbazepine acetate, ethosuximide, ethotoin, felbamate, fenfluramine, fosphenytoin, gabapentin, ganaxolone, huperzine A, lacosamide, lamotrigine, levetiracetam, nitrazepam, oxcarbazepine, perampanel, piracetam, phenobarbital, phenytoin, potassium bromide, pregabalin, primidone, retigabine, rufinamide, valproic acid, sodium valproate, stiripentol, tiagabine, topiramate, vigabatrin, or zonisamide. The AED may be valproic acid, sodium valproate, clonazepam, ethosuximide, felbamate, gabapentin, carbamazepine, oxcarbazepine, lamotrigine, levetiracetam, benzodiazepine, phenobarbital, pregabalin, primidone, tiagabine, topiramate, potassium bromide, phenytoin, stiripentol, vigabatrin, or zonisamide. The AED may be valproic acid, sodium valproate, gabapentin, topiramate, carbamazepine, oxcarbazepine, or vigabatrin.

The AED may be acetazolamide. The AED may be benzodiazepine. The AED may be cannabadiols. The AED may be carbamazepine. The AED may be clobazam. The AED may be clonazepam. The AED may be eslicarbazepine acetate. The AED may be ethosuximide. The AED may be ethotoin. The AED may be felbamate. The AED may be fenfluramine. The AED may be fosphenytoin. The AED may be gabapentin. The AED may be ganaxolone. The AED may be huperzine A. The AED may be lacosamide. The AED may be lamotrigine. The AED may be levetiracetam. The AED may be nitrazepam. The AED may be oxcarbazepine. The AED may be perampanel. The AED may be piracetam. The AED may be phenobarbital. The AED may be phenytoin. The AED may be potassium bromide. The AED may be pregabalin. The AED may be primidone. The AED may be retigabine. The AED may be rufinamide. The AED may be valproic acid. The AED may be sodium valproate. The AED may be stiripentol. The AED may be tiagabine. The AED may be topiramate. The AED may be vigabatrin. The AED may be zonisamide. Clemizole or a clemizole analog (including pharmaceutically acceptable salts thereof) or a pharmaceutical composition of clemizole or a clemizole analog may be administered as an adjunctive therapy to one or more of the AEDs described herein.

In methods described herein, the test compound may be co-administered with an anti-epileptic drug (AED). The AED may be acetazolamide, benzodiazepine, cannabadiols, carbamazepine, clobazam, clonazepam, eslicarbazepine acetate, ethosuximide, ethotoin, felbamate, fenfluramine, fosphenytoin, gabapentin, ganaxolone, huperzine A, lacosamide, lamotrigine, levetiracetam, nitrazepam, oxcarbazepine, perampanel, piracetam, phenobarbital, phenytoin, potassium bromide, pregabalin, primidone, retigabine, rufinamide, valproic acid, sodium valproate, stiripentol, tiagabine, topiramate, vigabatrin, or zonisamide. The AED may be valproic acid, sodium valproate, clonazepam, ethosuximide, felbamate, gabapentin, carbamazepine, oxcarbazepine, lamotrigine, levetiracetam, benzodiazepine, phenobarbital, pregabalin, primidone, tiagabine, topiramate, potassium bromide, phenytoin, stiripentol, vigabatrin, or zonisamide. The AED may be valproic acid, sodium valproate, gabapentin, topiramate, carbamazepine, oxcarbazepine, or vigabatrin.

The AED may be acetazolamide. The AED may be benzodiazepine. The AED may be cannabadiols. The AED may be carbamazepine. The AED may be clobazam. The AED may be clonazepam. The AED may be eslicarbazepine acetate. The AED may be ethosuximide. The AED may be ethotoin. The AED may be felbamate. The AED may be fenfluramine. The AED may be fosphenytoin. The AED may be gabapentin. The AED may be ganaxolone. The AED may be huperzine A. The AED may be lacosamide. The AED may be lamotrigine. The AED may be levetiracetam. The AED may be nitrazepam. The AED may be oxcarbazepine. The AED may be perampanel. The AED may be piracetam. The AED may be phenobarbital. The AED may be phenytoin. The AED may be potassium bromide. The AED may be pregabalin. The AED may be primidone. The AED may be retigabine. The AED may be rufinamide. The AED may be valproic acid. The AED may be sodium valproate. The AED may be stiripentol. The AED may be tiagabine. The AED may be topiramate. The AED may be vigabatrin. The AED may be zonisamide. Clemizole or a clemizole analog (including pharmaceutically acceptable salts thereof) or a pharmaceutical composition of clemizole or a clemizole analog may be administered as an adjunctive therapy to one or more of the AEDs described herein.

The test compound may thus be administered as an add-on (e.g. in combination with) AED medications for treating seizures, including seizures associated with the epilepsy disorders described herein. The test compound may be administered as an adjunctive therapy (e.g. in combination with) AED medications for treating seizures, including seizures associated with the epilepsy disorders described herein.

The epilepsy may be characterized by partial seizures or generalized seizures. The epilepsy may be characterized by partial seizures. The epilepsy may be characterized by generalized seizures. The partial seizure may be a simple focal seizure, a complex focal seizure, or a partial focal seizure with secondary generalization. The generalized seizure may be a generalized tonic-clonic seizure, an absence seizure (i.e. petit mal), a myoclonic seizure, a clonic seizure, a tonic seizure, or an atonic seizure.

When co-administered with the AEDs described herein, the test compound and the AED may be administered simultaneously. When administered simultaneously, the test compound may be formulated together with the AED (i.e. in a single dosage unit). The test compound may be formulated for separate administration from the AED but administered at the same time. When co-administered with AEDs described herein, the test compound may be administered sequentially (e.g. before or after) the administration of the AED. As set forth herein, one skilled in the art could readily determine the sequential order of administration.

Provided herein are methods of administering the test compound to the epileptic animal model and obtaining an electrophysiological recording of the animal model to detect the presence, intensity or absence of a spontaneous electrographic seizure in the animal model. The behavioral activity of the epileptic animal model may be a convulsive behavior, a convulsive high-velocity swim behavior or spontaneous electrographic seizures. In embodiments, the behavioral activity of the epileptic animal model may be a convulsive behavior. In embodiments, the behavioral activity of the epileptic animal model may be a convulsive high-velocity swim behavior. In embodiments, the behavioral activity of the epileptic animal model may be spontaneous electrographic seizures.

The convulsive high velocity swim behavior of the epileptic animal model can be detected with an electrophysiological recording of the animal model. The presence, intensity, or absence of a spontaneous electrographic seizure in said epileptic animal model can be detected with electrophysiological recording of the animal model.

In embodiments, measuring agonistic activity of a test compound may include measuring receptor binding activity of the test compound. In embodiments, the receptor may be a 5-HT_{2B} receptor. In embodiments, a test compound selected as a compound for treating epilepsy has low binding activity to 5-HT_{2A} receptor or 5-HT_{2C} receptor or does not demonstrate measurable 5-HT_{2A} receptor or 5-HT_{2C} receptor binding activity. In embodiments, the test compound selected has low binding activity to 5-HT_{2A} receptor or does not demonstrate measurable 5-HT_{2A} receptor binding activity. In embodiments, the test compound selected has low binding activity to 5-HT_{2C} receptor or does not demonstrate measurable 5-HT_{2C} receptor binding activity. In embodiments, the test compound selected has low binding activity to 5-HT_{2A} receptor. In embodiments, the test compound does not demonstrate measurable 5-HT_{2A} receptor binding activity. In embodiments, the test compound has low binding activity to 5-HT_{2C} receptor. In embodiments, the test compound does not demonstrate measurable 5-HT_{2C} receptor binding activity.

In embodiments, a test compound selected as a compound for treating epilepsy binds to 5-HT_{2B} receptor with a Kd of less than 100 nM, 10 nM, 1 nM, 500 pM, or 100 pM. In embodiments, the test compound selected binds to 5-HT_{2B} receptor with a Kd of less than 100 nM. In embodiments, the test compound selected binds to 5-HT_{2B} receptor with a Kd of less than 10 nM. In embodiments, the test compound selected binds to 5-HT_{2B} receptor with a Kd of less than 1 nM. In embodiments, the test compound selected binds to 5-HT_{2B} receptor with a Kd of less than 500 pM. In embodiments, the test compound selected binds to 5-HT_{2B} receptor with a Kd of less than 100 pM.

In embodiments, the test compound selected as a compound for treating epilepsy binds to 5-HT_{2B} receptor with a Kd of less than 50 nM, 25 nM, 15 nM, 7.5 nM, 5 nM, 2 nM, 800 pM, 600 pM, 400 pM, 200 pM, 150 pM, or 50 pM. In embodiments, the test compound selected binds to 5-HT_{2B} receptor with a Kd of less than 50 nM. In embodiments, the test compound selected binds to 5-HT_{2B} receptor with a Kd of less than 25 nM. In embodiments, the test compound selected binds to 5-HT_{2B} receptor with a Kd of less than 15 nM. In embodiments, the test compound selected binds to 5-HT_{2B} receptor with a Kd of less than 7.5 nM. In embodiments, the test compound selected binds to 5-HT_{2B} receptor with a Kd of less than 5 nM. In embodiments, the test compound selected binds to 5-HT_{2B} receptor with a Kd of less than 2 nM. In embodiments, the test compound selected binds to 5-HT_{2B} receptor with a Kd of less than 800 pM. In embodiments, the test compound selected binds to 5-HT_{2B} receptor with a Kd of less than 600 pM. In embodiments, the test compound selected binds to 5-HT_{2B} receptor with a Kd of less than 400 pM. In embodiments, the test compound selected binds to 5-HT_{2B} receptor with a Kd of less than 200 pM. In embodiments, the test compound selected binds to 5-HT_{2B} receptor with a Kd of less than 1500 pM. In embodiments, the test compound selected binds to 5-HT_{2B} receptor with a Kd of less than 50 pM.

In embodiments, the agonistic activity of the test compound selected as a compound for treating epilepsy to 5-HT_{2B} receptor is 10, 100, 1000, 10000, or 100000 times greater relative to 5-HT_{2A} receptor agonistic activity of said compound. In embodiments, the agonistic activity of the selected compound to 5-HT_{2B} receptor is 10 times greater relative to 5-HT_{2A} receptor agonistic activity of said compound. In embodiments, the agonistic activity of the selected compound to 5-HT_{2B} receptor is 100 times greater relative to 5-HT_{2A} receptor agonistic activity of said compound. In embodiments, the agonistic activity of the selected compound to 5-HT_{2B} receptor is 1000 times greater relative to 5-HT_{2A} receptor agonistic activity of said compound. In embodiments, the agonistic activity of the selected compound to 5-HT_{2B} receptor is 10000 times greater relative to 5-HT_{2A} receptor agonistic activity of said compound. In embodiments, the agonistic activity of the selected compound to 5-HT_{2B} receptor is 100000 times greater relative to 5-HT_{2A} receptor agonistic activity of said compound.

In embodiments, the agonistic activity of the selected compound to 5-HT_{2B} receptor is 50 times greater relative to 5-HT_{2A} receptor agonistic activity of said compound. In embodiments, the agonistic activity of the selected compound to 5-HT_{2B} receptor is 500 times greater relative to 5-HT_{2A} receptor agonistic activity of said compound. In embodiments, the agonistic activity of the selected compound to 5-HT_{2B} receptor is 2500 times greater relative to 5-HT_{2A} receptor agonistic activity of said compound. In embodiments, the agonistic activity of the selected compound to 5-HT_{2B} receptor is 5000 times greater relative to 5-HT_{2A} receptor agonistic activity of said compound. In embodiments, the agonistic activity of the selected compound to 5-HT_{2B} receptor is 25000 times greater relative to 5-HT_{2A} receptor agonistic activity of said compound. In embodiments, the agonistic activity of the selected compound to 5-HT_{2B} receptor is 50000 times greater relative to 5-HT_{2A} receptor agonistic activity of said compound. In embodiments, the agonistic activity of the selected compound to 5-HT_{2B} receptor is 1000000 times greater relative to 5-HT_{2A} receptor agonistic activity of said compound.

In embodiments, the compound selected by conducting a method of selecting a compound for treating epilepsy herein is a test compound having at least one of (1) 5-HT_{2B} agonistic activity, (2) reduced epileptic behavioral activity in the epileptic animal model after administering the test compound to the epileptic animal model, (3) reduction in convulsive high-velocity swim behavior in the epileptic animal model after administering the test compound to the epileptic animal model, (4) detected low intensity or absence of a spontaneous electrographic seizure in the epileptic animal model after administering the test compound to the epileptic animal model, (5) binding to 5-HT_{2B} receptor with a Kd of less than 100 nM, 10 nM, 1 nM, 500 pM, or 100 pM, or (6) agonistic activity to 5-HT_{2B} that is 10, 100, 1000, 10000, or 100000 times greater relative to 5-HT_{2A} receptor agonistic activity of said compound.

In embodiments, the compound selected by a method of selecting a compound for treating epilepsy herein is a test compound having 5-HT_{2B} agonistic activity. In embodiments, said HT-5_{2B} agonistic activity is HT-5_{2B} receptor binding activity. In embodiments, said compound has low binding activity to 5-HT_{2A} receptor or 5-HT_{2C} receptor or does not demonstrate measurable 5-HT_{2A} receptor or 5-HT_{2C} receptor binding activity. In embodiments, the HT-52B agonistic activity of the selected compound is 10, 100, 1000, 10000, or 100000 times greater relative to 5-HT_{2A} receptor agonistic activity of said compound. In embodiments, the HT-52B agonistic activity of the selected compound is 10 times greater relative to 5-HT_{2A} receptor agonistic activity of said compound. In embodiments, the HT-5_{2B} agonistic activity of the selected compound is 100 times greater relative to 5-HT_{2A} receptor agonistic activity of said compound. In embodiments, the HT-5_{2B} agonistic activity of the selected compound is 1000 times greater relative to 5-HT_{2A} receptor agonistic activity of said compound. In embodiments, the HT-5_{2B} agonistic activity of the selected compound is 10000 times greater relative to 5-HT_{2A} receptor agonistic activity of said compound. In embodiments, the HT-5_{2B} agonistic activity of the selected compound is 100000 times greater relative to 5-HT_{2A} receptor agonistic activity of said compound.

In embodiments, the compound selection selected by a method of selecting a compound for treating epilepsy herein is a test compound causing a reduced epileptic behavioral activity in the epileptic animal model after administering the test compound to the epileptic animal model. In embodiments, the epileptic behavioral activity is convulsive high-velocity swim behavior or spontaneous electrographic seizure. In embodiments, the epileptic behavioral activity is convulsive high-velocity swim behavior. In embodiments, the epileptic behavioral activity is spontaneous electrographic seizure.

In embodiments, the compound selected by a method of selecting a compound for treating epilepsy herein is a test compound causing a reduction in convulsive high-velocity swim behavior in the epileptic animal model after administering the test compound to the epileptic animal model.

In embodiments, the compound selected by a method of selecting a compound for treating epilepsy herein is a test compound discovered by detecting a low intensity or absence of a spontaneous electrographic seizure in the epileptic animal model after administering the test compound to the epileptic animal model. In embodiments, the test compound selection is based on detecting a low intensity of a spontaneous electrographic seizure in the epileptic animal model after administering the test compound to the epileptic animal model. In embodiments, the test compound selection is based on absence of a spontaneous electrographic seizure in the epileptic animal model after administering the test compound to the epileptic animal model.

In embodiments, the compound selected by a method of selecting a compound for treating epilepsy herein is a test compound that binds to 5-HT_{2B} receptor with a Kd of less than 100 nM, 10 nM, 1 nM, 500 pM, or 100 pM. In embodiments, the selected compound binds to 5-HT_{2B} receptor with a Kd of less than 100 nM. In embodiments, the selected compound binds to 5-HT_{2B} receptor with a Kd of less than 10 nM. In embodiments, the selected compound binds to 5-HT_{2B} receptor with a Kd of less than 1 nM. In embodiments, the selected compound binds to 5-HT_{2B} receptor with a Kd of less than 500 pM. In embodiments, the selected compound binds to 5-HT_{2B} receptor with a Kd of less than 100 pM.

Provided herein is a method of selecting a compound for treating epilepsy, said method includes, contacting a test compound with 5-HT_{2B} receptor, measuring the 5-HT_{2B} agonistic activity of the test compound, administering the test compound to an epileptic animal model, and measuring the behavioral activity in said epileptic animal model. In embodiments, the epileptic animal model may be a scn1lab mutant zebrafish. In embodiments, the agonistic activity of the test compound is measured by one or more of: convulsive high-velocity swim behavior in the scn1lab mutant zebrafish, spontaneous electrographic seizures in the scn1lab mutant zebrafish, or 5-HT_{2B} binding activity of the test compound. In embodiments, the agonistic activity of the test compound is measured by convulsive high-velocity swim behavior in the scn1lab mutant zebrafish. In embodiments, the agonistic activity of the test compound is measured by spontaneous electrographic seizures in the scn1lab mutant zebrafish. In embodiments, the agonistic activity of the test compound is measured by its 5-HT_{2B} binding activity.

Provided herein is a method of treating an epilepsy. In one aspect, the method is a method of treating an epilepsy by administering to a subject in need thereof, an effective amount of a 5-HT_{2B} specific receptor agonist.

In embodiments, the 5-HT_{2B} specific receptor agonist is clemizole, a clemizole analog, or a pharmaceutically acceptable salt thereof. In embodiments, the 5-HT_{2B} specific receptor agonist is not clemizole, a clemizole analog, or a pharmaceutically acceptable salt thereof. In embodiments, the 5-HT_{2B} specific receptor agonist is methylergonovine, 6-APB, norfenfluramine, Ro60-0175, BW-723C86, cabergoline, bromocriptine, or piribedil. In embodiments, the 5-HT_{2B} specific receptor agonist is a compound selected as a compound for treating epilepsy through a method of selecting a compound for treating epilepsy herein.

The analog of clemizole may include compounds of formula (I) described herein and may include compounds of similar structure as set forth, for example, in PCT/US2008/076804, WO10107739, WO2009039248, or U.S. Pat. No. 4,011,322, which are incorporated herein by reference.

In embodiments, the agonistic activity of the 5-HT_{2B} specific receptor agonist is 10, 100, 1000, 10000, or 100000 times greater relative to 5-HT_{2A} receptor activity of said 5-HT_{2B} specific receptor agonist. In embodiments, the agonistic activity of the 5-HT_{2B} specific receptor agonist is 10 times greater relative to 5-HT_{2A} receptor activity of said 5-HT_{2B} specific receptor agonist. In embodiments, the agonistic activity of the 5-HT_{2B} specific receptor agonist is 100 times greater relative to 5-HT_{2A} receptor activity of said 5-HT_{2B} specific receptor agonist. In embodiments, the agonistic activity of the 5-HT_{2B} specific receptor agonist is 1000 times greater relative to 5-HT_{2A} receptor activity of said 5-HT_{2B} specific receptor agonist. In embodiments, the agonistic activity of the 5-HT_{2B} specific receptor agonist is 10000 times greater relative to 5-HT_{2A} receptor activity of said 5-HT_{2B} specific receptor agonist. In embodiments, the agonistic activity of the 5-HT_{2B} specific receptor agonist is 100000 times greater relative to 5-HT_{2A} receptor activity of said 5-HT_{2B} specific receptor agonist. The times greater activity may be a value selected from the range 10 to 100, 100 to 1000, 1000 to 10000, or 10000 to 100000.

In embodiments, the agonistic activity of the 5-HT_{2B} specific receptor agonist is 50, 500, 2500, 5000, 25000, 50000 or 1000000 times greater relative to 5-HT_{2A} receptor activity of said 5-HT_{2B} specific receptor agonist. In embodiments, the agonistic activity of the 5-HT_{2B} specific receptor agonist is 50 times greater relative to 5-HT_{2A} receptor activity of said 5-HT_{2B} specific receptor agonist. In embodiments, the agonistic activity of the 5-HT_{2B} specific receptor agonist is 500 times greater relative to 5-HT_{2A} receptor activity of said 5-HT_{2B} specific receptor agonist. In embodiments, the agonistic activity of the 5-HT_{2B} specific receptor agonist is 2500 times greater relative to 5-HT_{2A} receptor activity of said 5-HT_{2B} specific receptor agonist. In embodiments, the agonistic activity of the 5-HT_{2B} specific receptor agonist is 5000 times greater relative to 5-HT_{2A} receptor activity of said 5-HT_{2B} specific receptor agonist. In embodiments, the agonistic activity of the 5-HT_{2B} specific receptor agonist is 25000 times greater relative to 5-HT_{2A} receptor activity of said 5-HT_{2B} specific receptor agonist. In embodiments, the agonistic activity of the 5-HT_{2B} specific receptor agonist is 50000 times greater relative to 5-HT_{2A} receptor activity of said 5-HT_{2B} specific receptor agonist. In embodiments, the agonistic activity of the 5-HT_{2B} specific receptor agonist is 1000000 times greater relative to 5-HT_{2A} receptor activity of said 5-HT_{2B} specific receptor agonist. The times greater activity may be a value selected from the range 50 to 500, 500 to 2500, 2500 to 5000, 5000 to 25000, 25000 to 50000, or 50000 to 100000.

In embodiments, the 5-HT_{2B} specific receptor agonist binds to 5-HT_{2B} receptor with a Kd of less than 100 nM, 10 nM, 1 nM, 500 pM, or 100 pM. In embodiments, the 5-HT_{2B} specific receptor agonist binds to 5-HT_{2B} receptor with a Kd of less than 100 nM. In embodiments, the 5-HT_{2B} specific receptor agonist binds to 5-HT_{2B} receptor with a Kd of less than 10 nM. In embodiments, the 5-HT_{2B} specific receptor agonist binds to 5-HT_{2B} receptor with a Kd of less than 1 nM. In embodiments, the 5-HT_{2B} specific receptor agonist binds to 5-HT_{2B} receptor with a Kd of less than 500 pM. In embodiments, the 5-HT_{2B} specific receptor agonist binds to 5-HT_{2B} receptor with a Kd of less than 100 pM. The Kd may be a specific value in a range from 100 pM to 500 pM, 500 pM to 1 nM, 1 nM to 10 nM, or 10 nM to 100 nM. The specific value may be selected from any 1 pM increment in the range. The Kd may be selected from a sub-range within any of the aforementioned Kd ranges. The low endpoint of a sub-range may be the low end of the range or any value selected from 1 pM increments above the low end of the range up to 1 pM less that the high end of the range. The high endpoint of a sub-range may be the high end of the range or any value selected from 1 pM below the high end of the range to 1 pM greater than the low end of the range.

In embodiments, the 5-HT_{2B} specific receptor agonist binds to 5-HT_{2B} receptor with a Kd of less than 50 nM, 25 nM, 15 nM, 7.5 nM, 5 nM, 2 nM, 800 pM, 600 pM, 400 pM, 200 pM, 150 pM, or 50 pM. In embodiments, the 5-HT_{2B} specific receptor agonist binds to 5-HT_{2B} receptor with a Kd of less than 50 nM. In embodiments, the 5-HT_{2B} specific receptor agonist binds to 5-HT_{2B} receptor with a Kd of less than 25 nM. In embodiments, the 5-HT_{2B} specific receptor agonist binds to 5-HT_{2B} receptor with a Kd of less than 15 nM. In embodiments, the 5-HT_{2B} specific receptor agonist binds to 5-HT_{2B} receptor with a Kd of less than 7.5 nM. In embodiments, the 5-HT_{2B} specific receptor agonist binds to 5-HT_{2B} receptor with a Kd of less than 5 nM. In embodiments, the 5-HT_{2B} specific receptor agonist binds to 5-HT_{2B} receptor with a Kd of less than 2 nM. In embodiments, the 5-HT_{2B} specific receptor agonist binds to 5-HT_{2B} receptor with a Kd of less than 800 pM. In embodiments, the 5-HT_{2B} specific receptor agonist binds to 5-HT_{2B} receptor with a Kd of less than 600 pM. In embodiments, the 5-HT_{2B} specific receptor agonist binds to 5-HT_{2B} receptor with a Kd of less than 400 pM. In embodiments, the 5-HT_{2B} specific receptor agonist binds to 5-HT_{2B} receptor with a Kd of less than 200 pM. In embodiments, the 5-HT_{2B} specific receptor agonist binds to 5-HT_{2B} receptor with a Kd of less than 1500 pM. In embodiments, the 5-HT_{2B} specific receptor agonist binds to 5-HT_{2B} receptor with a Kd of less than 50 pM. The Kd may be a specific value in a range from 50 pM to 150 pM, 150 pM to 200 pM, 200 pM to 400 pM, 400 pM to 600 pM, 600 pM to 800 pM, 800 pM to 2 nM, 2 nM to 5 nM, 5 nM to 7.5 nM, 7.5 nM to 15 nM, 15 nM to 25 nM, or 25 nM to 50 nM. The specific value may be selected from any 1 pM increment in the range. The Kd may be selected from a sub-range within any of the aforementioned Kd ranges. The low endpoint of a sub-range may be the low end of the range or any value selected from 1 pM increments above the low end of the range up to 1 pM less that the high end of the range. The high endpoint of a sub-range may be the high end of the range or any value selected from 1 pM below the high end of the range to 1 pM greater than the low end of the range.

### II. Pharmaceutical Compositions

Provided herein are pharmaceutical compositions comprising a 5-HT_{2B} receptor agonist or a pharmaceutically acceptable salt thereof useful for treating the aforementioned diseases and disorders. In embodiments, the 5-HT_{2B} specific receptor agonist is a compound selected as a compound for treating epilepsy through a method of selecting a compound for treating epilepsy herein. The pharmaceutical composition may be formulated as a tablet, a powder, a capsule, a pill, a cachet, or a lozenge as described herein. The pharmaceutical composition may be formulated as a tablet, capsule, pill, cachet, or lozenge for oral administration. The pharmaceutical composition may be formulated for dissolution into a solution for administration by such techniques as, for example, intravenous administration. The pharmaceutical composition may be formulated for oral administration, suppository administration, topical administration, intravenous administration, intraperitoneal administration, intramuscular administration, intralesional administration, intrathecal administration, intranasal administration, subcutaneous administration, implantation, transdermal administration, or transmucosal administration as described herein.

When administered as pharmaceutical composition, the pharmaceutical compositions may include optical isomers, diastereomers, enantiomers, isoforms, polymorphs, hydrates, solvates or products, or pharmaceutically acceptable salts of the 5-HT_{2B} receptor agonist. The 5-HT_{2B} receptor agonist (including pharmaceutically acceptable salts thereof) included in the pharmaceutical composition may be covalently attached to a carrier moiety, as described above. In embodiments, the 5-HT_{2B} receptor agonist (including pharmaceutically acceptable salts thereof) included in the pharmaceutical composition is not covalently linked to a carrier moiety. A combination of covalently and not covalently linked 5-HT_{2B} receptor agonist may be in a pharmaceutical composition herein.

The 5-HT_{2B} receptor agonist (including pharmaceutically acceptable salts thereof) may be administered alone or co-administered to a subject in need thereof with an AED as described herein. Co-administration is meant to include simultaneous or sequential administration as described herein of the 5-HT_{2B} receptor agonist individually or in combination (e.g. more than one compound—e.g. an AED described herein). The preparations can also be combined, when desired, with other active substances (e.g. to prevent seizures).

1. Formulations

The 5-HT_{2B} receptor agonist (including pharmaceutically acceptable salts thereof) or a pharmaceutical composition described herein can be prepared and administered in a wide variety of oral, parenteral, and topical dosage forms. Thus, the 5-HT_{2B} receptor agonist (including pharmaceutically acceptable salts thereof) or a pharmaceutical composition described herein may be in a formulation for injection, and may be administered by injection (e.g. intravenously, intramuscularly, intracutaneously, subcutaneously, intraduodenally, or intraperitoneally). Also, the 5-HT_{2B} receptor agonist (including pharmaceutically acceptable salts thereof) or a pharmaceutical composition described herein may be in a formulation for inhalation, and may be administered by inhalation. The inhalation may be intranasal. Additionally, the 5-HT_{2B} receptor agonist (including pharmaceutically acceptable salts thereof) or a pharmaceutical composition may be in a formulation for transdermal delivery, and may be administered transdermally. It is also envisioned that multiple routes of administration (e.g., intramuscular, oral, transdermal) may be used to administer the 5-HT_{2B} receptor agonist (including pharmaceutically acceptable salts thereof) or a pharmaceutical composition comprising same. The pharmaceutical compositions described herein may include a pharmaceutically acceptable carrier or excipient and one or more of a 5-HT_{2B} receptor agonist (including pharmaceutically acceptable salts thereof). The pharmaceutical compositions described herein may include a pharmaceutically acceptable carrier or excipient, one or more of a 5-HT_{2B} receptor agonist (including pharmaceutically acceptable salts thereof) and one or more AED as described herein.

Preparation may include pharmaceutically acceptable carriers. The pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of the 5-HT_{2B} receptor agonist (including pharmaceutically acceptable salts thereof) or a pharmaceutical composition (i.e., dosage). Pharmaceutical preparations described herein can also be used orally using, for example, push-fit capsules made of gelatin, as well as soft, sealed capsules made of gelatin and a coating such as glycerol or sorbitol.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for the 5-HT_{2B} receptor agonist (including pharmaceutically acceptable salts thereof) or a pharmaceutical composition comprising same are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The 5-HT_{2B} receptor agonist (including pharmaceutically acceptable salts thereof) or a pharmaceutical composition comprising same can also be incorporated into liposomes or administered via transdermal pumps or patches. Pharmaceutical admixtures suitable for use herein include those described, for example, in Pharmaceutical Sciences (17th Ed., Mack Pub. Co., Easton, Pa.) and WO 96/05309, the teachings of both of which are hereby incorporated by reference.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an allylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included herein are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations described herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg according to the particular application and the potency of the active component. The composition can, if desired, also contain other compatible therapeutic agents.

Formulations may include a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity-building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, combinations of the foregoing, and other agents known to those skilled in the art. Such agents are typically employed at a level between about 0.01% and about 2% by weight. Determination of acceptable amounts of any of the above adjuvants is readily ascertained by one skilled in the art.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

The 5-HT_{2B} receptor agonist (including pharmaceutically acceptable salts thereof) or a pharmaceutical composition thereof may be delivered transdermally, for treating the epilepsy disorders described herein, by a topical route, formulated as applicator sticks, solutions, suspensions, emulsions, gels, creams, ointments, pastes, jellies, paints, powders, and aerosols.

The 5-HT_{2B} receptor agonist (including pharmaceutically acceptable salts thereof) may be provided as a salt in the pharmaceutical compositions described herein and can be formed with many acids, including but not limited to hydrochloric, sulfuric, acetic, lactic, tartaric, malic, succinic, etc. Salts tend to be more soluble in aqueous or other protonic solvents that are the corresponding free base forms.

The 5-HT_{2B} receptor agonist (including pharmaceutically acceptable salts thereof) or a pharmaceutical composition thereof administered for treating epilepsy disorders described herein may be administered via parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

The pharmaceutical formulations of the 5-HT_{2B} receptor agonist (including pharmaceutically acceptable salts thereof) for treating an epilepsy disorder may be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

Co-administration includes administering one active agent (e.g. clemizole or a clemizole analog (including pharmaceutically acceptable salts thereof)) within 0.5, 1, 2, 4, 6, 8, 10, 12, 16, 20, or 24 hours of a second active agent (e.g. an anticonvulsant). Co-administration may include administering one active agent within 0.5, 1, 2, 4, 6, 8, 10, 12, 16, 20, or 24 hours of a second active agent. Co-administration may include administering two active agents simultaneously, approximately simultaneously (e.g., within about 1, 5, 10, 15, 20, or 30 minutes of each other), or sequentially in any order. Co-administration can be accomplished by co-formulation, i.e., preparing a single pharmaceutical composition including both active agents. In other embodiments, the active agents can be formulated separately. The active and/or adjunctive agents may be linked or conjugated to one another.

Co-administration also includes combination with treatments for epilepsy disorders such as dietary requirements or dietary changes. Accordingly, the 5-HT_{2B} receptor agonist (including pharmaceutically acceptable salts thereof) or a pharmaceutical composition thereof may be administered to subjects on specialized diets, including but not limited to, a ketogenic diet (e.g. a high-fat, adequate-protein, low-carbohydrate diet).

2. Effective Dosages

The pharmaceutical composition may include the 5-HT_{2B} receptor agonist (including pharmaceutically acceptable salts thereof) contained in a therapeutically effective amount, i.e., in an amount effective to achieve its intended purpose. The actual amount effective for a particular application will depend, inter alia, on the condition being treated. For example, when administered in methods to treat an epilepsy disorder (e.g. Dravet Syndrome), such compositions will contain amounts of the 5-HT_{2B} receptor agonist (including pharmaceutically acceptable salts thereof) or a pharmaceutical composition thereof effective to achieve the desired result (e.g. inhibiting seizures, which may include reducing severity of or elimination seizures).

The dosage and frequency (single or multiple doses) of the 5-HT_{2B} receptor agonist (including pharmaceutically acceptable salts thereof) or a pharmaceutical composition thereof administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods described herein.

The therapeutically effective amounts of the 5-HT_{2B} receptor agonist (including pharmaceutically acceptable salts thereof) or a pharmaceutical composition thereof for treating epilepsy diseases described herein may be initially determined from cell culture assays. Target concentrations will be those concentrations of the 5-HT_{2B} receptor agonist (including pharmaceutically acceptable salts thereof) or a pharmaceutical composition thereof capable of inhibiting or otherwise decreasing seizures experienced by a patient.

Therapeutically effective amounts of the 5-HT_{2B} receptor agonist (including pharmaceutically acceptable salts thereof) or a pharmaceutical composition thereof for use in humans may be determined from animal models. For example, a dose for humans can be formulated to achieve a concentration that has been found to be effective in animals. The dosage in humans can be adjusted by monitoring response of the patient to the treatment and adjusting the dosage upwards or downwards, as described above.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered 5-HT_{2B} receptor agonist (including pharmaceutically acceptable salts thereof) or a pharmaceutical composition thereof effective for the particular epilepsy disorder being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of the 5-HT_{2B} receptor agonist (including pharmaceutically acceptable salts thereof) or a pharmaceutical composition thereof by considering factors such as potency, relative bioavailability, patient body weight, presence and severity of adverse side effects, preferred mode of administration, and the toxicity profile of the selected agent.

3. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the compound is used.

When parenteral application is needed or desired, particularly suitable admixtures for the 5-HT_{2B} receptor agonist (including pharmaceutically acceptable salts thereof) included in the pharmaceutical composition may be injectable, sterile solutions, oily or aqueous solutions, as well as suspensions, emulsions, or implants, including suppositories. Non limiting examples of carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, Pa.) and WO 96/05309, the teachings of both of which are hereby incorporated by reference.

### EMBODIMENTS

Embodiment 1. A method of selecting a compound for use in treating epilepsy, said method comprising: contacting a test compound with 5-hydroxytryptamine-2B receptor (5-HT_{2B}); and measuring the 5-HT_{2B} agonistic activity of said test compound.

Embodiment 2. The method of embodiment 1, further comprising an epileptic animal model.

Embodiment 3. The method of embodiment 1 or 2, further comprising administering said test compound to the epileptic animal model and measuring a behavioral activity in said epileptic animal model.

Embodiment 4. The method of any one of embodiments 1-3, wherein said epileptic animal model is a Dravet Syndrome (DS) animal model.

Embodiment 5. The method of any one of embodiments 1-4, wherein the DS animal model is a zebrafish (Danio rerio) that is resistant to anti-epilepsy drugs (AEDs).

Embodiment 6. The method of any one of embodiments 1-5, wherein the zebrafish (Danio rerio) is an scn1lab mutant or an scn1laa mutant.

Embodiment 7. The method of any one of embodiments 1-6, wherein the behavioral activity is a convulsive high-velocity swim behavior.

Embodiment 8. The method of any one of embodiments 2-7, further comprising administering said test compound to the epileptic animal model and obtaining an electrophysiological recording of the animal model to detect the presence, intensity, or absence of a spontaneous electrographic seizure in said epileptic animal model.

Embodiment 9. The method of any one of embodiments 1-8, wherein said epileptic animal model is the DS animal model.

Embodiment 10. The method of any one of embodiments 1-9, wherein the DS animal model is the scn1lab mutant zebrafish.

Embodiment 11. The method of any one of embodiments 1-10, wherein said measuring comprises determining 5-HT_{2B} receptor binding activity by the test compound.

Embodiment 12. The method of any one of embodiments 1-11, wherein said compound has low binding activity to 5-HT_{2A} receptor or 5-HT₂C receptor or does not demonstrate measurable 5-HT_{2A} receptor or 5-HT₂C receptor binding activity.

Embodiment 13. The method of any one of embodiments 1-12, wherein said compound binds to 5-HT_{2B} receptor with a Kd of less than 100 nM, 10 nM, 1 nM, 500 pM, or 100 pM.

Embodiment 14. The method of any one of embodiments 1-13, wherein the agonistic activity of said compound to 5-HT_{2B} receptor is 10, 100, 1000, 10000, or 100000 times greater relative to 5-HT_{2A} receptor agonistic activity of said compound.

Embodiment 15. The method of any one of embodiments 1-14, further comprising selecting said test compound as said compound based on at least one of (1) 5-HT_{2B} agonistic activity of said test compound, (2) reduced epileptic behavioral activity in said epileptic animal model after administering said test compound to said epileptic animal model, (3) reduction in convulsive high-velocity swim behavior in said epileptic animal model after administering said test compound to said epileptic animal model, (4) detecting a low intensity or absence of a spontaneous electrographic seizure in said epileptic animal model after administering said test compound to said epileptic animal model, (5) binding of said test compound to 5-HT_{2B} receptor with a Kd of less than 100 nM, 10 nM, 1 nM, 500 pM, or 100 pM, or (6) agonistic activity of said test compound that is 10, 100, 1000, 10000, or 100000 times greater relative to 5-HT_{2A} receptor agonistic activity of said compound.

Embodiment 16. A method of selecting a compound for treating an epilepsy, said method comprising:

contacting a test compound with 5-HT_{2B} receptor;

measuring the 5-HT_{2B} agonistic activity of said test compound;

administering said test compound to an epileptic animal model; and

measuring a behavioral activity in said epileptic animal model.

Embodiment 17. The method of embodiment 16, wherein the epileptic animal model is a scn1lab mutant zebrafish and the agonistic activity of said test compound is measured by one or more of:

convulsive high-velocity swim behavior in the scn1lab mutant zebrafish;

spontaneous electrographic seizures in the scn1lab mutant zebrafish; or

5-HT_{2B} binding activity of said test compound.

Embodiment 18. A method of treating an epilepsy in a subject in need thereof, said method comprising administering to said subject an effective amount of a 5-HT_{2B} specific receptor agonist.

Embodiment 19. The method of embodiment 18, wherein the agonistic activity of said 5-HT_{2B} specific receptor agonist is 10 times greater relative to 5-HT_{2A} receptor agonistic activity of said 5-HT_{2B} specific receptor agonist.

Embodiment 20. The method of embodiment 18, wherein the agonistic activity of said 5-HT_{2B} specific receptor agonist is 100 times greater relative to 5-HT_{2A} receptor agonistic activity of said 5-HT_{2B} specific receptor agonist.

Embodiment 21. The method of embodiment 18, wherein the agonistic activity of said 5-HT_{2B} specific receptor agonist is 1000 times greater relative to 5-HT_{2A} receptor agonistic activity of said 5-HT_{2B} specific receptor agonist.

Embodiment 22. The method of embodiment 21, wherein the agonistic activity of said 5-HT_{2B} specific receptor agonist is 10000 times greater relative to 5-HT_{2A} receptor agonistic activity of said 5-HT_{2B} specific receptor agonist.

Embodiment 23. The method of embodiment 22, wherein the agonistic activity of said 5-HT_{2B} specific receptor agonist is 100000 times greater relative to 5-HT_{2A} receptor agonistic activity of said 5-HT_{2B} specific receptor agonist.

Embodiment 24. The method of any one of embodiments 18 to 23, wherein said 5-HT_{2B} specific receptor agonist binds to 5-HT_{2B} receptor with a Kd of less than 100 nM.

Embodiment 25. The method of any one of embodiments 18 to 23, wherein said 5-HT_{2B} specific receptor agonist binds to 5-HT_{2B} receptor with a Kd of less than 10 nM.

Embodiment 26. The method of any one of embodiments 18 to 23, wherein said 5-HT_{2B} specific receptor agonist binds to 5-HT_{2B} receptor with a Kd of less than 1 nM.

Embodiment 27. The method of any one of embodiments 18 to 23, wherein said 5-HT_{2B} specific receptor agonist binds to 5-HT_{2B} receptor with a Kd of less than 500 pM.

Embodiment 28. The method of any one of embodiments 18 to 23, wherein said 5-HT_{2B} specific receptor agonist binds to 5-HT_{2B} receptor with a Kd of less than 100 pM.

### EXAMPLES

### Chemical Synthesis of Clemizole Analogs

Definition of abbreviations used: PPA=polyphosphoric acid: Ph=phenyl; K2CO3=potassium carbonate; EtOH=ethanol; NaH=sodium hydride; THF=tetrahydrofuran; TBAI=tetrabutylammonium idodide; LiOH=lithium hydroxide; MeOH=methanol; HATU—1-[Bis(dimethylamino)methylene]-1H-1,2,3-triazolo[4,5-b]pyridinium 3-oxid hexafluorophosphate, N-[(Dimethylamino)-1H-1,2,3-triazolo-[4,5-b]pyridin-1-ylmethylene]-N-methylmethanaminium hexafluorophosphate N-oxide; DIEA=N,N-diisopropylethyl amine; DMF=dimethylformamide; Na(OAc)₃BH=sodium triacetoxyborohydride; HCl=hydrochloric acid; rt=room temperature; LLw=microwave treatment.

General Synthesis of Clemizole Analogs 1-28

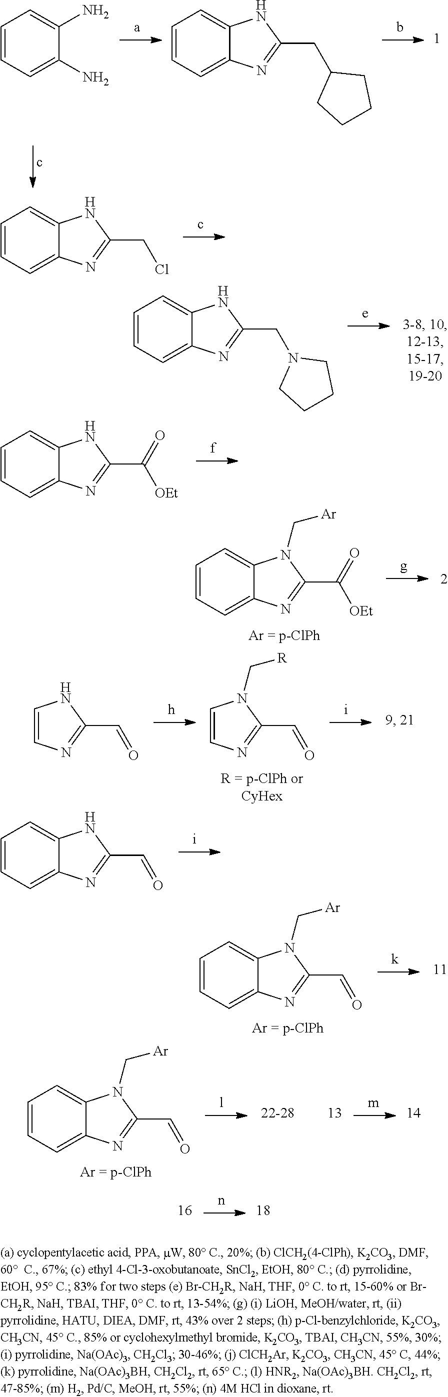

Unless otherwise noted all chemical reagents and solvents used are commercially available. Air and/or moisture sensitive reactions were carried out under an argon atmosphere in oven-dried glassware using anhydrous solvents from commercial suppliers. Air and/or moisture sensitive reagents were transferred via syringe or cannula and were introduced into reaction vessels through rubber septa. Solvent removal was accomplished with a rotary evaporator at ca. 10-50 Torr. 1H NMR spectra were recorded on a Varian INOVA-400 400 MHz spectrometer. Chemical shifts are reported in 6 units (ppm). NMR spectra were referenced relative to residual NMR solvent peaks. Coupling constants (J) are reported in hertz (Hz). Microwave reactions were carried out in a CEM Discover microwave reactor. Column chromatography was carried out using Isolera Four flash chromatography system and SiliaSep silica gel cartridges from Silicycle. LC/MS data were acquired on a Waters Micromass ZQ mass spectrometer equipped with Waters 2795 Separation Module, Waters 2424 Evaporative Light Scattering Detector and Waters 2996 Photodiode Array Detector. Separations were carried out with XTerra® MS C18, 5 μm, 4.6×50 mm column, at ambient temperature (unregulated) using a mobile phase of water-methanol containing a constant 0.1% formic acid.

Compounds were commercially sourced from Millipore Sigma (Methylergonovine maleate, BW-723C86, 1-(3-Chlorophenyl)piperazine hydrochloride (m-CPP), (+)-Norfenfluramine hydrochloride), Cayman Chemicals (Cabergoline, Bromocriptine mesylate, (−)-Apomorphine hydrochloride), ApexBio (Ro 60-0175 fumarate), Tocris Bioscience (CP-809,101 hydrochloride), AK Scientific, Inc (Piribedil), and Axon Medchem (TL 99 hydrobromide). 10 mM compound stock solutions were made in DMSO and then diluted in embryo medium for assays.

### Example 1: 1-[(4-Chlorophenyl)methyl]-2-(cyclopentylmethyl)-1H-benzimidazole (1)

Step 1: A mixture of o-phenylenediamine (0.25 g, 2.3 mmol), cyclopentylacetic acid (0.29 ml, 2.3 mmol) and polyphosphoric acid (1.0 ml) were heated in a microwave reactor at 80° C. for 30 minutes. The reaction mixture was diluted with ethyl acetate, washed with aqueous saturated sodium bicarbonate solution, water and brine. The organic layer was dried over magnesium sulfate, concentrated and purified by flash column chromatography (50% ethyl acetate/hexanes) to obtain 2-(cyclopentylmethyl)-1H-benzimidazole as a light brown solid (93 mg, 20% yield). ¹H NMR (400 MHz, CDCl3) δ 7.57 (dd, J=6.1, 3.2 Hz, 2H), 7.29-7.19 (m, 2H), 2.95 (d, J=7.5 Hz, 2H), 2.48-2.38 (m, 1H), 1.96-1.78 (m, 2H), 1.72-1.50 (m, 4H), 1.36-1.23 (m, 2H); LC-MS (m/z) for C₁₃H₁₇N₂⁺ [M+H]⁺: calculated 201.13, found 200.99.

Step 2: 4-Chlorobenzyl chloride (0.04 g, 0.2 mmol) was added to a mixture of 2-(cyclopentylmethyl)-1H-benzimidazole (0.05 g, 0.2 mmol) and potassium carbonate (0.069 g, 0.5 mmol) in N,N-dimethylformamide (2 mL). After stirring at 60° C. for 3 h, the reaction mixture was diluted with ethyl acetate, washed with water and brine. The organic layer was dried over magnesium sulfate, concentrated and purified by flash column chromatography (25% ethyl acetate/hexanes) to obtain 1-[(4-chlorophenyl)methyl]-2-(cyclopentylmethyl)-1H-benzimidazole (1) as a white solid (55 mg, 67% yield). ¹H NMR (400 MHz, CDCl3) δ 7.79 (d, J=7.8 Hz, 1H), 7.33-7.15 (m, 5H), 6.98 (d, J=8.3 Hz, 2H), 5.35 (s, 2H), 2.85 (d, J=7.5 Hz, 2H), 2.51-2.39 (m, 1H), 1.85 (td, J=11.5, 6.9 Hz, 2H), 1.73-1.53 (m, 4H), 1.35-1.22 (m, 2H); LC-MS (m/z) C₂₀H₂₂ClN₂⁺ [M+H]⁺: calculated 325.14, found 324.98.

### Example 2: 1-[(4-Chlorophenyl)methyl]-2-(pyrrolidine-1-carbonyl)-1H-benzimidazole (2)

Step 1: A mixture of 1H-benzimidazole-2-carboxylic acid ethyl ester (0.1 g, 0.5 mmol) and pyrrolidine (0.216 ml, 2.6 mmol) were heated to 130° C. in the microwave reactor for 30 minutes. The reaction mixture was concentrated and azeotrope dried with toluene to obtain about 115 mg of 2-(pyrrolidine-1-carbonyl)-1H-benzimidazole as a brown solid which was used without further purification.

Step 2: 4-Chlorobenzyl chloride (0.03 g, 0.2 mmol) was added to a mixture of sodium hydride, 60% (0.005 g, 0.2 mmol) and 2-(pyrrolidine-1-carbonyl)-1H-benzimidazole (0.04 g, 0.2 mmol) in N,N-dimethylformamide (1 mL). After stirring at room temperature for 3 h, the reaction mixture was quenched with aqueous saturated ammonium chloride and extracted with ethyl acetate. The organic extracts were washed with water and brine. The organic layer was dried over magnesium sulfate, concentrated and purified by flash column chromatography (0-50% ethyl acetate/hexanes) to obtain 1-[(4-chlorophenyl)methyl]-2-(pyrrolidine-1-carbonyl)-1H-benzimidazole (2) as a clear oil (29 mg, 46% yield). ¹H NMR (400 MHz, CDCl3) δ 7.89-7.82 (m, 1H), 7.37-7.24 (m, 7H), 5.71 (s, 2H), 3.90 (br t, J=6.0 Hz, 2H), 3.67 (br t, J=6.3 Hz, 2H), 2.01-1.90 (m, 4H); LC-MS (m/z) for C₁₉H₁₉ClN₃O⁺ [M+H]⁺: calculated 340.12, found 340.21.

### Example 3: 1-(Cyclohexylmethyl)-2-[(pyrrolidin-1-yl)methyl]-1H-benzimidazole (4)

Step 1: Tin(II) chloride, dihydrate (0.209 g, 0.9 mmol) was added to a mixture of o-phenylenediamine (1.0 g, 9.2 mmol) and ethyl 4-chloro-3-oxobutanoate (1.25 ml, 9.2 mmol) in ethanol (20 mL) and heated to 80° C. for 2 h. The reaction mixture was concentrated to remove ethanol, washed with hexanes and dried to obtain about 1.7 g of crude 2-(chloromethyl)-1H-benzimidazole as a yellow solid which was used without further purification.

Step 2: A mixture of 2-(chloromethyl)-1H-benzimidazole (1.5 g, 9.0 mmol) and pyrrolidine (14.8 ml, 180.1 mmol) in ethanol (20 mL) was heated to 95° C. for 2 h and room temperature for 72 h. The reaction mixture was concentrated and purified by flash column chromatography (0-10% methanol/dichloromethane with 10% 7 N ammonia in methanol) to obtain 2-[(pyrrolidin-1-yl)methyl]-1H-benzimidazole as a reddish-brown solid (1.5 g, 83% yield). ¹H NMR (400 MHz, CDCl3) δ 7.65-7.53 (m, 2H), 7.31-7.20 (m, 2H), 4.17 (s, 2H), 3.50 (s, 2H), 2.94-2.83 (m, 4H), 1.93 (br s, 4H); LC-MS (m/z) for C₁₂H₁₆N₃⁺ [M+H]⁺: calculated 202.13, found 202.07.

Step 3: Cyclohexylmethyl bromide (0.038 ml, 0.3 mmol) and tetrabutylammonium iodide (0.009 g, 0.025 mmol) were added to a cooled (0° C.) mixture of 2-[(pyrrolidin-1-yl)methyl]-1H-benzimidazole (0.05 g, 0.25 mmol) and sodium hydride, 60% (0.007 g, 0.3 mmol) in tetrahydrofuran (1 mL). The reaction mixture was heated to 50° C. for 2 h and then diluted with ethyl acetate, washed with 10% aqueous ammonium hydroxide solution, water and brine. The organic layer was dried over magnesium sulfate, concentrated and purified by flash column chromatography (5% methanol/dichloromethane) to obtain 1-(cyclohexylmethyl)-2-[(pyrrolidin-1-yl)methyl]-1H-benzimidazole (4) as a reddish-brown oil (39 mg, 52% yield). ¹H NMR (400 MHz, CDCl3) δ 7.85-7.68 (m, 1H), 7.38-7.20 (m, 3H), 4.17 (d, J=7.3 Hz, 2H), 3.95 (s, 2H), 2.66-2.54 (m, 4H), 1.96-1.63 (m, 10H), 1.30-1.03 (m, 5H); LC-MS (m/z) for C₁₉H₂₈N₃⁺ [M+H]⁺: calculated 298.22, found 298.06.

### Example 4: 2-[(Pyrrolidin-1-yl)methyl]-1-{[4-(trifluoromethyl)phenyl]methyl}-1H-benzimidazole (6)

4-(Trifluoromethyl)benzyl bromide (0.038 ml, 0.2 mmol) was added to a cooled (0° C.) mixture of 2-[(pyrrolidin-1-yl)methyl]-1H-benzimidazole (0.05 g, 0.2 mmol) and sodium hydride, 60% (0.007 g, 0.3 mmol) in tetrahydrofuran (1 mL). The reaction mixture was stirred at room temperature for 2 h and then diluted with ethyl acetate, washed with 10% aqueous ammonium hydroxide solution, water and brine. The organic layer was dried over magnesium sulfate, concentrated and purified by flash column chromatography (5% methanol/dichloromethane) & (50% acetone/dichloromethane) to obtain 2-[(pyrrolidin-1-yl)methyl]-1-{[4-(trifluoromethyl)phenyl]methyl}-1H-benzimidazole (6) as a pale yellow oil (25 mg, 28% yield) that solidifies on standing. ¹H NMR (400 MHz, CDCl3) δ 7.81 (d, J=7.3 Hz, 1H), 7.57 (d, J=8.3 Hz, 2H), 7.34-7.18 (m, 5H), 5.67 (s, 2H), 3.90 (s, 2H), 2.61-2.49 (m, 4H), 1.77-1.61 (m, 4H); LC-MS (m/z) for C₂₀H₂₁F₃N₃⁺ [M+H]⁺: calculated 360.16, found 360.05.

### Example 5: 1-[(4-Chlorophenyl)methyl]-2-[(pyrrolidin-1-yl)methyl]-1H-imidazole (9)

Step 1: 1H-Imidazole-2-carbaldehyde (0.25 g, 2.6 mmol), 4-chlorobenzyl chloride (0.5 g, 3.1 mmol) & potassium carbonate (0.72 g, 5.2 mmol) were stirred in acetonitrile (20 mL) at 45° C. for 18 h. The reaction mixture was diluted with ethyl acetate, washed with water and brine. The organic layer was dried over magnesium sulfate, concentrated and purified by flash column chromatography (50% ethyl acetate/hexanes) to obtain 1-[(4-chlorophenyl)methyl]-1H-imidazole-2-carbaldehyde as a pale green oil (0.49 g, 85% yield). ¹H NMR (400 MHz, CDCl3) δ 9.82 (s, 1H), 7.32-7.26 (m, 3H), 7.16-7.10 (m, 3H), 5.56 (s, 2H); LC-MS (m/z) for C₁₁H₁₀ClN₂O⁺ [M+H]⁺: calculated 221.04, found 220.83.

Step 2: Sodium triacetoxyborohydride (0.053 g, 0.2 mmol) was added to a mixture of 1-[(4-chlorophenyl)methyl]-1H-imidazole-2-carbaldehyde (0.05 g, 0.2 mmol) and pyrrolidine (0.019 ml, 0.2 mmol) stirring in dichloromethane (2 mL). After stirring at room temperature for 18 h, the reaction mixture was washed with 10% aqueous ammonium hydroxide solution, water and brine. The organic layer was dried over magnesium sulfate, concentrated and purified by flash column chromatography (0-10% methanol/dichloromethane/10% 7N ammonia in methanol) to obtain 1-[(4-chlorophenyl)methyl]-2-[(pyrrolidin-1-yl)methyl]-1H-imidazole (9) as a yellow solid (19 mg, 30% yield). ¹H NMR (400 MHz, CDCl3) δ 7.36-7.26 (m, 2H), 7.08 (d, J=8.3 Hz, 2H), 6.99 (d, J=1.2 Hz, 1H), 6.86 (d, J=1.2 Hz, 1H), 5.27 (s, 2H), 3.67 (s, 2H), 2.55-2.46 (m, 4H), 1.85-1.67 (m, 4H); LC-MS (m/z) for C₁₅H₁₉ClN₃⁺ [M+H]⁺: calculated 276.12, found 276.01.

### Example 6: 1-[(4-Chlorophenyl)methyl]-2-[(pyrrolidin-1-yl)methyl]-1H-indole (11)

Step 1: 4-Chlorobenzyl chloride (0.133 g, 0.8 mmol) was added to a mixture of 1H-indole-2-carbaldehyde (0.1 g, 0.7 mmol) and potassium carbonate (0.19 g, 1.4 mmol) in acetonitrile (5 mL). After stirring at 45° C. for 18 h, the reaction mixture was diluted with ethyl acetate, washed with water and brine. The organic layer was dried over magnesium sulfate, concentrated and purified by flash column chromatography (25% ethyl acetate/hexanes) to obtain 1-[(4-chlorophenyl)methyl]-1H-indole-2-carbaldehyde as an orange colored solid (81 mg, 43% yield). ¹H NMR (400 MHz, CDCl3) δ 9.92 (s, 1H), 7.80 (d, J=7.8 Hz, 1H), 7.45-7.35 (m, 3H), 7.30-7.19 (m, 3H), 7.05 (d, J=8.5 Hz, 2H), 5.82 (s, 2H); LC-MS (m/z) for C₁₆H₁₃ClNO⁺ [M+H]⁺: calculated 270.06, found 269.96.

Step 2: Sodium triacetoxyborohydride (0.043 g, 0.2 mmol) was added to a cooled (0° C.) mixture of 1-[(4-chlorophenyl)methyl]-1H-indole-2-carbaldehyde (0.05 g, 0.2 mmol) and pyrrolidine (0.015 ml, 0.2 mmol) in dichloromethane. After stirring at room temperature for 18 h, the reaction mixture was washed with 10% aqueous ammonium hydroxide solution, water and brine. The organic layer was dried over magnesium sulfate, concentrated and purified by flash column chromatography (50% ethyl acetate/hexanes) to obtain 1-[(4-chlorophenyl)methyl]-2-[(pyrrolidin-1-yl)methyl]-1H-indole (11) as a pale yellow oil (39 mg, 65% yield). ¹H NMR (400 MHz, CDCl3) δ 7.70-7.51 (m, 1H), 7.32-7.09 (m, 5H), 6.96 (d, J=8.3 Hz, 2H), 6.46 (s, 1H), 5.53 (s, 2H), 3.68 (s, 2H), 2.58-2.41 (m, 4H), 1.80-1.66 (m, 4H); LC-MS (m/z) for C₂₀H₂₂ClN₂⁺ [M+H]⁺: calculated 325.14, found 325.02.

### Example 7: 1-(Cyclopentylmethyl)-2-[(pyrrolidin-1-yl)methyl]-1H-benzimidazole (20)

Bromomethyl cyclopentane (0.092 ml, 0.7 mmol) and tetrabutylammonium iodide (0.009 g, 0.02 mmol) were added to a mixture of 2-[(pyrrolidin-1-yl)methyl]-1H-benzimidazole (0.05 g, 0.2 mmol) and sodium hydride, 60% (0.009 g, 0.4 mmol) in N,N-dimethylformamide (1 mL). After stirring at 55° C. for 18 h, the reaction mixture was diluted with ethyl acetate, washed with water and brine. The organic layer was dried over magnesium sulfate, concentrated and purified by flash column chromatography (0-10% methanol/dichloromethane) to obtain 1-(cyclopentylmethyl)-2-[(pyrrolidin-1-yl)methyl]-1H-benzimidazole (20) as a reddish brown oil (35 mg, 50% yield). ¹H NMR (400 MHz, CDCl3) δ 7.85-7.66 (m, 1H), 7.41-7.22 (m, 3H), 4.28 (d, J=7.8 Hz, 2H), 3.96 (s, 2H), 2.64-2.44 (m, 5H), 1.83-1.54 (m, 10H), 1.42-1.24 (m, 2H); LC-MS (m/z) for C₁₈H₂₆N₃⁺ [M+H]⁺: calculated 284.21, found 284.01.

### Example 8: ({1-[(4-Chlorophenyl)methyl]-1H-1,3-benzodiazol-2-yl}methyl)diethylamine (24)

Sodium triacetoxyborohydride (0.043 g, 0.2 mmol) was added to a mixture of 1-(4-chlorobenzyl)-1H-benzimidazole-2-carbaldehyde (0.05 g, 0.18 mmol) and diethylamine (0.019 mL, 0.18 mmol) in dichloromethane (2 mL). After stirring at room temperature for 18 h, the reaction mixture was washed with water and brine. The organic layer was dried over magnesium sulfate, concentrated and purified by flash column chromatography (0-100% ethyl acetate/hexanes) to obtain ({1-[(4-chlorophenyl)methyl]-1H-1,3-benzodiazol-2-yl}methyl)diethylamine (24) as a reddish-brown oil (50 mg, 82% yield). ¹H NMR (400 MHz, CDCl3) δ 7.80 (d, J=7.6 Hz, 1H), 7.33-7.17 (m, 5H), 7.02 (d, J=8.8 Hz, 2H), 5.64 (s, 2H), 3.84 (s, 2H), 2.57 (q, J=7.1 Hz, 4H), 1.00 (t, J=7.2 Hz, 6H); LC-MS (m/z) for C₁₉H₂₃C₁N₃⁺ [M+H]⁺: calculated 328.15, found 327.98.

### Example 9: tert-Butyl 4-({1-[(4-chlorophenyl)methyl]-1H-benzimidazol-2-yl}methyl)piperazine-1-carboxylate (25)

Sodium triacetoxyborohydride (0.043 g, 0.2 mmol) was added to a mixture of 1-(4-chlorobenzyl)-1H-benzimidazole-2-carbaldehyde (0.05 g, 0.18 mmol) and tert-butyl 1-piperazinecarboxylate (0.034 g, 0.18 mmol) in dichloromethane (2 mL). After stirring at room temperature for 18 h, the reaction mixture was washed with water and brine. The organic layer was dried over magnesium sulfate, concentrated and purified by flash column chromatography (0-100% ethyl acetate/hexanes) to obtain tert-butyl 4-({1-[(4-chlorophenyl)methyl]-1H-benzimidazol-2-yl}methyl)piperazine-1-carboxylate (25) as a foamy pale yellow oil (59 mg, 72% yield). ¹H NMR (400 MHz, CDCl3) δ 7.79 (d, J=7.4 Hz, 1H), 7.34-7.19 (m, 6H), 7.03 (d, J=8.3 Hz, 2H), 5.55 (s, 2H), 3.77 (s, 2H), 3.33 (br s, 4H), 2.45 (br s, 4H), 1.46 (s, 9H); LC-MS (m/z) for C₂₄H₃₀ClN₄O₂⁺ [M+H]⁺: calculated 441.20, found 441.08.

Phenotypic screening of clemizole analogs. Twenty-eight clemizole analogs, Table 1A, were screened for efficacy in suppressing the high-velocity seizure-like swim behavior observed in scn1lab mutant zebrafish.

TABLE 1A
clemizole analogs library

| Compound | Structure | MW |
|---|---|---|
| 1 | 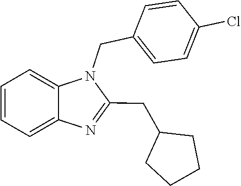 [SMILES: Clc1ccc(Cn2c(CC3CCCC3)nc3ccccc32)cc1] | 324.85 |
| 2 | 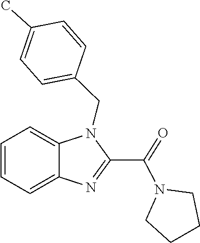 [SMILES: Cc1ccc(Cn2c(C(=O)N3CCCC3)nc3ccccc32)cc1] | 339.82 |
| 3 | 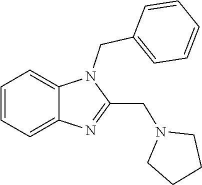 [SMILES: c1ccc(Cn2c(CN3CCCC3)nc3ccccc32)cc1] | 337.42 |
| 4 | 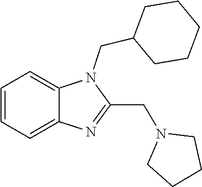 [SMILES: c1ccc2c(c1)nc(CN1CCCC1)n2CC1CCCCC1] | 297.44 |
| 5 | 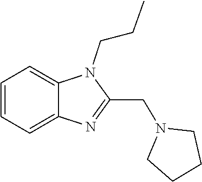 [SMILES: CCCn1c(CN2CCCC2)nc2ccccc21] | 289.37 |
| 6 | 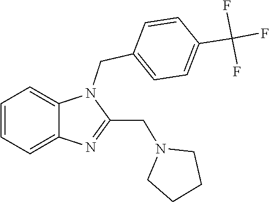 [SMILES: FC(F)(F)c1ccc(Cn2c(CN3CCCC3)nc3ccccc32)cc1] | 359.39 |
| 7 | 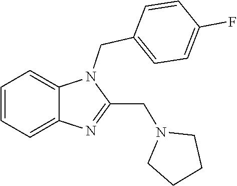 [SMILES: Fc1ccc(Cn2c(CN3CCCC3)nc3ccccc32)cc1] | 309.38 |
| 8 | 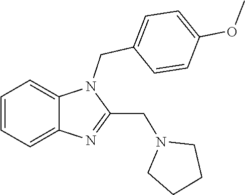 [SMILES: COc1ccc(Cn2c(CN3CCCC3)nc3ccccc32)cc1] | 367.44 |
| 9 | 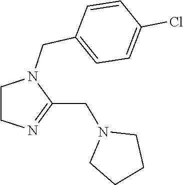 [SMILES: Clc1ccc(CN2CCN=C2CN2CCCC2)cc1] | 275.78 |
| 10 | 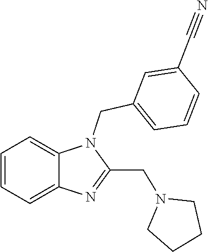 [SMILES: N#Cc1cccc(Cn2c(CN3CCCC3)nc3ccccc32)c1] | 316.40 |
| 11 | 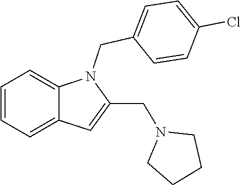 [SMILES: Clc1ccc(Cn2c(CN3CCCC3)cc3ccccc32)cc1] | 324.85 |
| 12 | 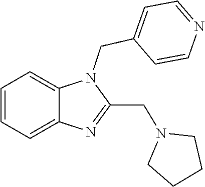 [SMILES: c1ccc2c(c1)nc(CN1CCCC1)n2Cc1ccncc1] | 384.43 |
| 13 | 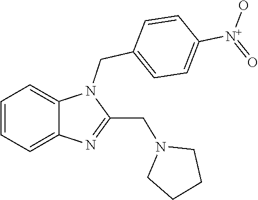 | 336.39 |
| 14 | 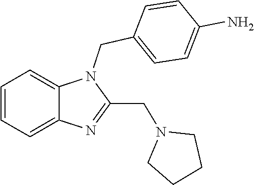 [SMILES: Nc1ccc(Cn2c(CN3CCCC3)nc3ccccc32)cc1] | 306.40 |
| 15 | 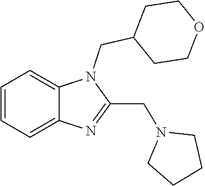 [SMILES: c1ccc2c(c1)nc(CN1CCCC1)n2CC1CCOCC1] | 299.41 |
| 16 | 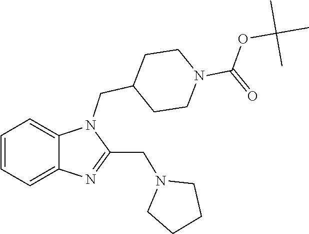 [SMILES: CC(C)(C)OC(=O)N1CCC(Cn2c(CN3CCCC3)nc3ccccc32)CC1] | 398.54 |
| 17 | 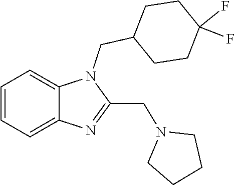 [SMILES: FC1(F)CCC(Cn2c(CN3CCCC3)nc3ccccc32)CC1] | 333.42 |
| 18 | 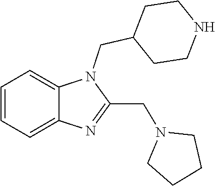 [SMILES: c1ccc2c(c1)nc(CN1CCCC1)n2CC1CCNCC1] | 371.35 |
| 19 | 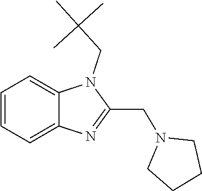 [SMILES: CC(C)(C)Cn1c(CN2CCCC2)nc2ccccc21] | 317.43 |
| 20 | 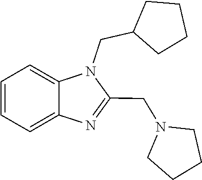 [SMILES: c1ccc2c(c1)nc(CN1CCCC1)n2CC1CCCC1] | 283.41 |
| 21 | 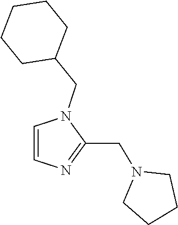 [SMILES: c1cn(CC2CCCCC2)c(CN2CCCC2)n1] | 339.43 |
| 22 | 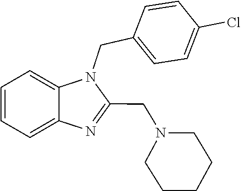 [SMILES: Clc1ccc(Cn2c(CN3CCCCC3)nc3ccccc32)cc1] | 339.86 |
| 23 | 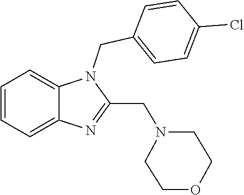 [SMILES: Clc1ccc(Cn2c(CN3CCOCC3)nc3ccccc32)cc1] | 341.83 |
| 24 | 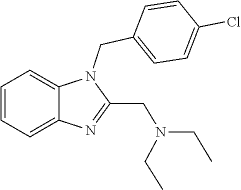 [SMILES: CCN(CC)Cc1nc2ccccc2n1Cc1ccc(Cl)cc1] | 327.85 |
| 25 | 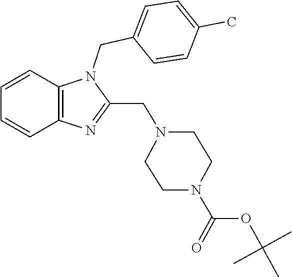 [SMILES: Cc1ccc(Cn2c(CN3CCN(C(=O)OC(C)(C)C)CC3)nc3ccccc32)cc1] | 440.97 |
| 26 | 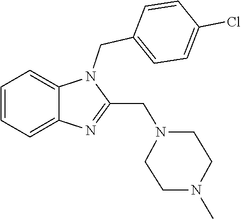 [SMILES: CN1CCN(Cc2nc3ccccc3n2Cc2ccc(Cl)cc2)CC1] | 354.88 |
| 27 | 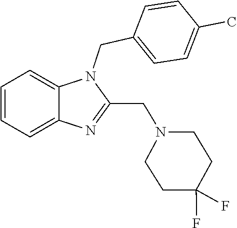 [SMILES: Cc1ccc(Cn2c(CN3CCC(F)(F)CC3)nc3ccccc32)cc1] | 375.84 |
| 28 | 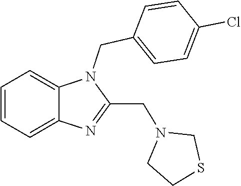 [SMILES: Clc1ccc(Cn2c(CN3CCSC3)nc3ccccc32)cc1] | 343.87 |

### Example 10

Epilepsy can be acquired as a result of an injury to the brain or genetic mutation. Among the genetic epilepsies more than 650 variants have been identified in the SCN1A gene (Harkin, L. A. et al. The spectrum of SCN1A-related infantile epileptic encephalopathies. Brain 130, 843-852 (2007); Mulley J. C., et al., SCN1A mutations and epilepsy. Hum. Mutat. 25, 535-542 (2005)). Missense or frame-shift mutations in this gene are associated with generalized epilepsy with febrile seizures plus (GEFS+) (Ceulemans, B. P., et al., Clinical correlations of mutations in the SCN1A gene: from febrile seizures to severe myoclonic epilepsy in infancy. Pediatric Neurol. 30, 236-243 (2004)) as well as a more severe disorder known as Dravet syndrome. Children with DS initially exhibit normal development but often experience febrile seizure episodes within the first year of life with eventual progression to severe spontaneous recurrent seizures, intellectual disability, ataxia, and psychomotor dysfunction. Seizures are inadequately managed using available antiepileptic drugs (AEDs) and these children are poor candidates for neurosurgical resection (Bender, A. C., et al., SCN1A mutations in Dravet syndrome: Impact of interneuron dysfunction on neural networks and cognitive outcome. Epilepsy Beh. 23, 177-186 (2012)).

In mammalian brain there are four main subtypes of voltage-gated sodium channel alpha subunits: Na_{V}1.1, Na_{V}1.2, Na_{V}1.3 and Na_{V}1.6, encoded for by the genes SCN1A, SCN2A, SCN3A, and SCN8A, respectively. Opening of these channels produces a sodium conductance and rapid cell membrane depolarization e.g., features integral to action potential initiation (Catterall, W. A., et al., Na_{V}1.1 channels and epilepsy. J. Physiol. 588, 1849-1859 (2010)). In mice, Na_{V}1.1 is widely expressed in the central nervous system including the axon initial segment of parvalbumin-positive hippocampal interneurons and excitatory principal cells (Kim, D. Y., et al., Reduced sodium channel Na_{V}1.1 levels in BACE1-null mice. J. Biol. Chem. 286, 8106-8116 (2011); Chen, C., et al., Mice lacking sodium channel beta1 subunits display defects in neuronal excitability, sodium channel expression, and nodal architecture. J. Neurosci. 24, 4030-4042 (2004)). Heterozygous deletion of Na_{V}1.1 in mice leads to a reduction in the firing capability of acutely dissociated fast-spiking interneurons (Yu, F. H., et al., Reduced sodium current in GABAergic interneurons in a mouse model of severe myoclonic epilepsy in infancy. Nat. Neurosci. 9, 1142-1149 (2006)). Mice with global or interneuron-specific heterozygous deletion of Na_{V}1.1 exhibit temperature-induced and spontaneous seizures, mild ataxia, autism-like behaviors and premature death (Yu, F. H., et al., Reduced sodium current in GABAergic interneurons in a mouse model of severe myoclonic epilepsy in infancy. Nat. Neurosci. 9, 1142-1149 (2006); Oakley, J. C., et al., Temperature- and age-dependent seizures in a mouse model of severe myoclonic epilepsy in infancy. Proc. Natl. Acad. Sci. USA 106, 3994-3999 (2009); Cheah, C. S., et al., Specific deletion of Na_{V}1.1 sodium channels in inhibitory interneurons causes seizures and premature death in a mouse model of Dravet syndrome. Proc. Natl. Acad. Sci. USA 109, 14646-14651 (2012)). Knock-in mouse carrying a premature stop codon in domain III of the Na_{V}1.1 channel also exhibit a decrement in spike amplitude during prolonged interneuron firing and increased sensitivity to temperature-induced seizures (Ogiwara, I., et al., Na_{V}1.1 localizes to axons of parvalbumin-positive inhibitory interneurons: a circuit basis for epileptic seizures in mice carrying an Scn1a gene mutation. J. Neurosci. 27, 5903-5914 (2007)).

Generation and characterization of valid animal models is critical to efforts to understand the pathophysiology of DS, and to aid in identification of novel therapies. While considerable attention has focused on modeling SCN1A mutations in mice these animals have proven difficult to breed and epilepsy phenotypes are strongly influenced by background strain genetics. Induced pluripotent stem cells can be generated from DS patients but individual neurons do not recapitulate the network environment necessary for in vivo seizure generation. Danio rerio (zebrafish), a simple vertebrate species, provide an alternative model system with significant advantages for genetic manipulation, cost-efficient breeding and in vivo drug discovery (Lessman, C. A., The developing zebrafish (Danio rerio): a vertebrate model for high-throughput screening of chemical libraries. Birth Defects Res. C. Embryo Today 93, 268-280 (2011); Delvecchio, C., et al., The zebrafish: a powerful platform for in vivo, HTS drug discovery. Assay Drug Dev. Technol. 9, 354-361 (2011); Rinkwitz, S., et al., Zebrafish: an integrative system for neurogenomics and neurosciences. Prog. Neurobiol. 93, 231-243 (2011)). Ideally, an animal model should be based on a known genetic cause of the disease (SCN1A mutation), accurately recapitulate key features of the disease (epilepsy), and respond, or not, to therapies commonly used in patients with the disease (pharmacological validation). If successful, such a model could inform the understanding of the disease process and catalyze explorations toward new therapies.

In zebrafish, the voltage-gated sodium channel family consists of four sets of duplicated genes: scn1Laa & scn1Lab, scn4aa & scn4ab, scn5Laa & scn5Lab, and scn8aa & scn8ab (Novak, A. E., et al., Embryonic and larval expression of zebrafish voltage-gated sodium channel alpha-subunit genes. Dev. Dyn. 235, 1962-1973 (2006)). The zebrafish scn1Lab gene shares a 77% identity with human SCN1A and is expressed in the central nervous system. A homozygous zebrafish mutant for this gene (originally termed didy^{s552}) was discovered in a chemical mutagenesis screen using the optokinetic response as an assay (Schoonheim, P. J., Arrenberg, A. B., Del Bene, F., & Baier H., Optogenetic localization and genetic perturbation of saccade-generating neurons in zebrafish. J. Neurosci. 30, 7111-7120 (2010)). These types of screens are based on inducing random point mutations using the alkylating agent N-ethyl-N-nitrosourea (ENU), resulting mutations are typically loss-of-function and recessive. Although this is a homozygous mutation, scn1Lab zebrafish mutants are relevant for the autosomal dominant human Dravet Syndrome given the genome duplication in zebrafish and the presence of an additional Na_{V}1.1 homologue (scn1Laa). scn1Lab mutants were characterized at the molecular and behavioral level, demonstrated that mutants exhibit spontaneous drug-resistant seizures, and then used them in a novel high-throughput screening program to identify compounds that ameliorate the epilepsy phenotype. A phenotype-based screen identified clemizole, an FDA-approved compound, as an effective inhibitor of spontaneous convulsive behaviors and electrographic seizures in these mutants. Clemizole analogs were then synthesized to test their effectiveness in ameliorating epilepsy.

Zebrafish maintenance. Zebrafish were raised under standard laboratory conditions in accordance with requirements outlined in the Guide for the Care and Use of Animals (ebrary Inc., 2011) and experiments were approved by the Institutional Animal Care and Use Committee (protocol #AN108659-03). Embryos were obtained by natural spawning of scn1lab (didy^{s552}) heterozygous animals that had been outcrossed to TL strain. Homozygous scn1lab mutants (n=2500) have dispersed melanosomes and appear visibly darker by 3 dpf compared to WT larvae.

Seizure monitoring. At 5 dpf individual zebrafish larvae were placed into a single well of a clear flat-bottomed 96-well microplate containing embryo media. Larvae were selected randomly as sex determination is not possible at this stage of development. All drug screening experiments were conducted in an unbiased manner by investigators blinded to the test compounds and all files coded for post hoc analysis. Microplates were placed inside the DanioVision and acclimated for 20 min at room temperature. Locomotion plots were obtained for each well at a recording epoch of 10 min using EthoVision XT software (DanioVision, Noldus Information Technology). Seizure scoring was performed using the following three-stage scale established for pentylenetetrazole-induced seizures: Stage 0, no or very little swim activity; Stage I, increased, brief bouts of swim activity; Stage II, rapid “whirlpool-like” circling swim behavior; and Stage III, paroxysmal whole-body clonus-like convulsions, and a brief loss of posture. WT fish are typically scored at Stage 0 or I. Plots were analyzed for distance travelled (in millimeters) and mean velocity (in millimeters per second). After 90 mins of drug exposure larvae were examined for toxic side-effects. Compounds that decreased or stopped the larva heartbeat, or reduced or eliminated the escape response when touched, were considered toxic.

For electrophysiology studies, zebrafish larvae were anesthetized with cold and immobilized in 1.2% agarose; local field potential (LFP) recordings were obtained from forebrain or optic tectum structures using a single-electrode approach, as previously described (Baraban, S. C. et al., Neuroscience, 131, 759-768, 2005). Agarose-embedded LFP recording sessions of 10 min were obtained at 1 kHz. Epileptiform events were identified post hoc and defined as multi-spike or poly-spike upward or downward membrane deflections greater than 3× baseline noise level and 150-250 ms in duration (interictal-like) or greater than 5× baseline noise, multi-spike and >500 ms in duration (ictal-like); both events were counted using threshold detection settings in Clampfit (Molecular Devices; Sunnyvale, Calif.). During electrophysiology experiments larvae were continuously monitored for blood flow and heart rate using an Axiocam digital camera at video frame rate.

Receptor Binding Assays. In vitro binding assay and Ki data were performed by the US National Institute of Mental Health Psychoactive Drug Screening Program (NIMH PDSP). Drugs were screened against recombinant, stably expressed human 5-HT_{2A}R, 5-HT_{2B}R, 5-HT_{2C}R and H₁. For specific binding assays please refer to detailed procedures at https://pdspdb.unc.edu/pdspWeb/ (Besnard, J. et al., Nature, 492, 215-220, 2012).

Statistics. Data are presented as the mean±standard error of the mean (SEM), unless otherwise stated. For behavior analysis, the threshold for a change in mean swim velocity ≥40% (>1.5×SD of 250 control treated scn1lab) is considered significant. For comparison between more than two groups one-way ANOVA analysis was used. When variance did not have a normal distribution the non-parametric Kruskal-Wallis test was used followed by Dunns multiple comparison test. Differences considered statistically significant are indicated with asterisks (*P<0.05; **P<0.01).

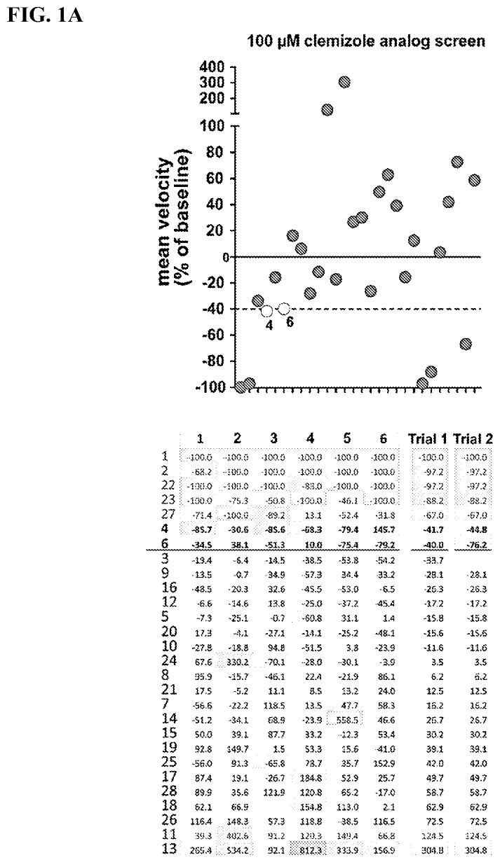

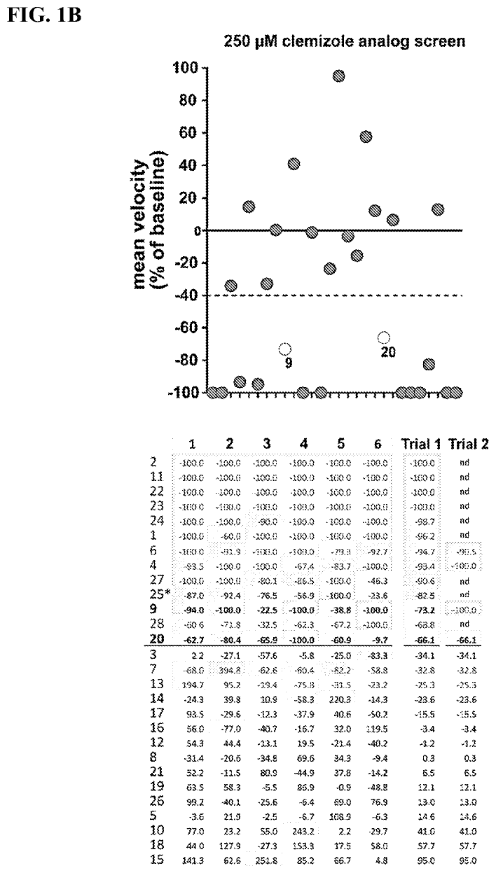

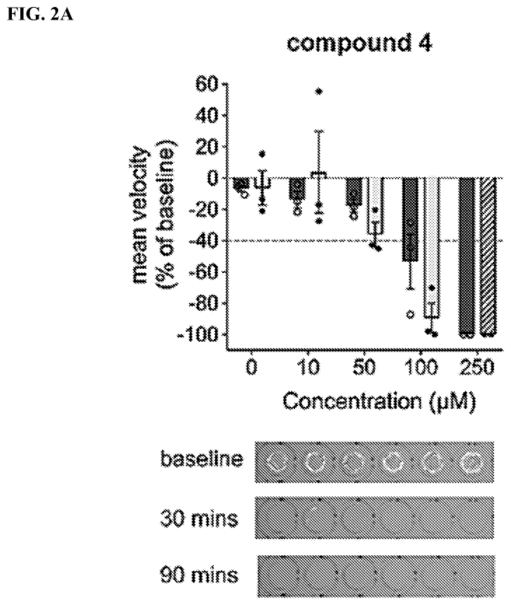

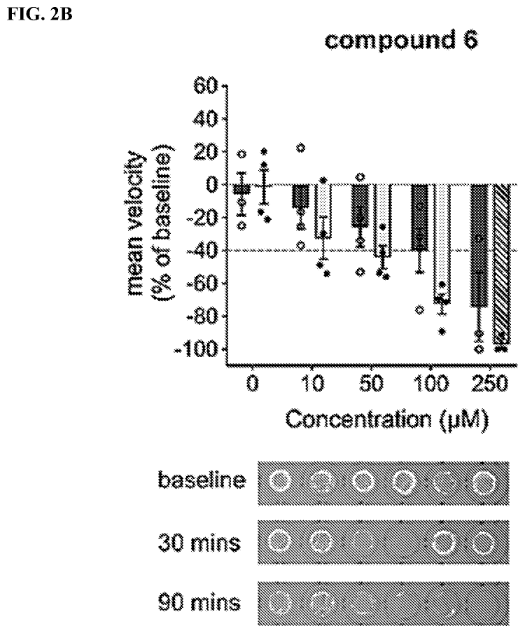

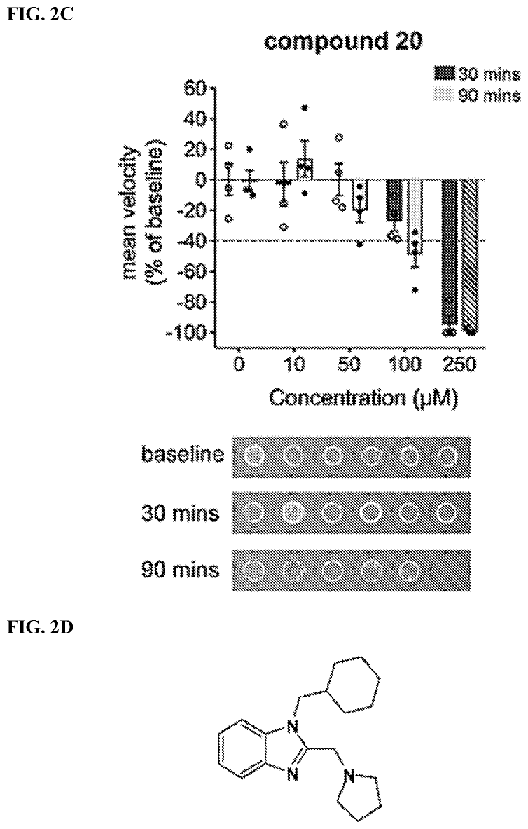

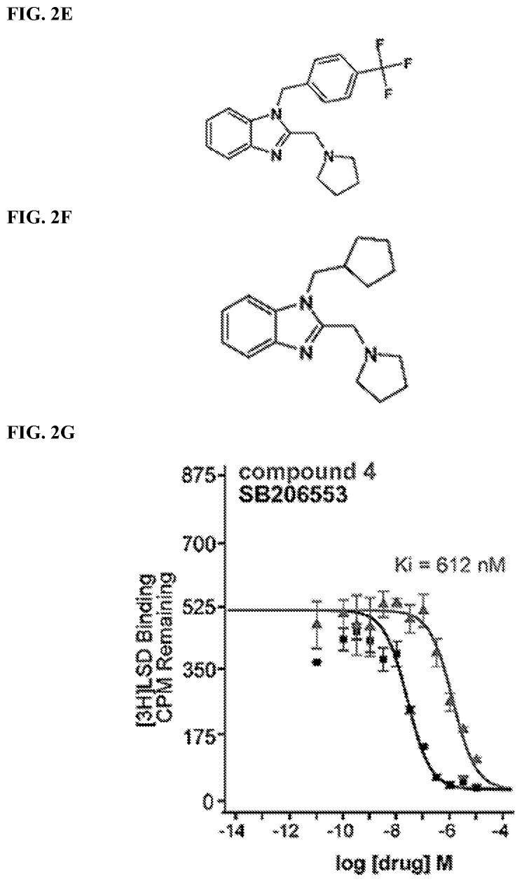

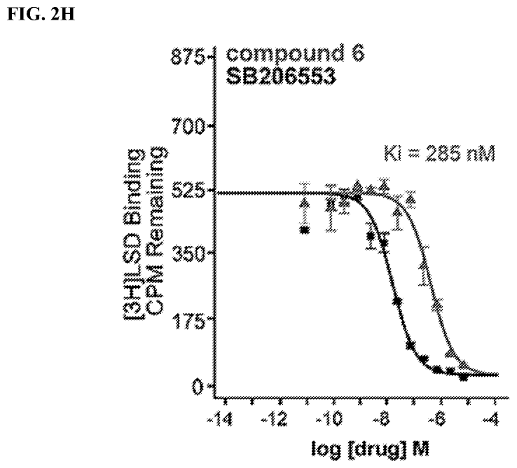

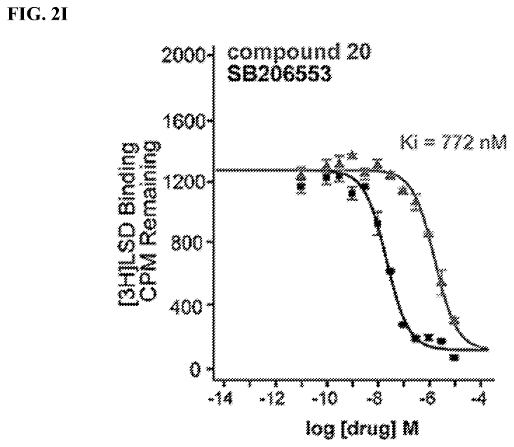

Evaluation of clemizole analogs that reduce seizure-like swim behavior in scn1lab mutant zebrafish. Plots show the change in mean swim velocity of 5 dpf larvae screened at (FIG. 1A) 100 μM, or (FIG. 2B) 250 μM (six fish per drug treatment). Threshold for inhibition of seizure activity (positive hits—labeled data points) was determined as a reduction in mean swim velocity of ≥40% (dashed line). The red data points represent compounds that were classified as toxic after 90-min exposure. The heat map shows the % change in velocity for the six individual larva from the first trial (1-6). Mean velocity change from six individual fish is shown for trial 1 and 2.

The screening of clemizole analogs revealed that five compounds (17.9%) were toxic at 100 μM, which increased to 12 compounds (42.9%) at 250 μM. The clemizole analogs 4, 6, 9, and 20 were effective in suppressing the scn1lab mutant seizure-like behavior at 100 μM and 250 μM, respectively. Clemizole analog 25 (*) failed to go into solution at 250 μM so it was not considered for further testing.

To confirm the anti-seizure effect on convulsive behaviors of 5 dpf scn1lab mutants, clemizole analogs 4, 6, 9 and 20 were synthesized independently (by Oxygen Healthcare Research Pvt. Ltd.) using the same methodology as described herein. Newly synthesized compounds were retested at 10, 50, 100 and 250 μM to confirm a concentration-dependent response. Clemizole analogs 4, 6 and 20 reduced the high-velocity seizure-like swim behavior observed in the scn1lab mutant zebrafish larvae, confirming the initial screening results with analogs synthesized at UCSF (FIG. 2A-C). The threshold for a decrease in velocity is ≥40% (dashed line). Locomotion of larvae was recorded for 10 min after an exposure of 30 min (black bars) and 90 min (gray bars). A representative raw 10 min tracking plot is shown for a single experiment of six individual scn1Lab zebrafish. In vitro radioligand binding analyses of (FIG. 2G) compound 4, (FIG. 2H) compound 6 and (FIG. 2I) compound 20 revealed specificity for 5-HT_{2B}R over other 5-HT₂R subtypes. Compound SB206553 was used as a positive control for 5-HT_{2B}R binding. Binding affinity for other clemizole analogs are given in Table 1B and Table 2. The resynthesized compound 9 was toxic at 250 μM. This confirmed the result from the second testing of the original compound and suggests the decrease in swim behavior observed during trial 1 may be a false positive result.

TABLE 1B
Receptor specificity and binding affinity (Ki) for compounds effective in
suppressing spontaneous seizure activity in scn1lab mutant zebrafish.

| | Comp. 4 | Comp. 6 | Comp. 20 | MeERGO | 6-APB | CLEM | TRAZ | LOR | FEN |
|---|---|---|---|---|---|---|---|---|---|
| | (nM) | (nM) | (nM) | (nM) | (nM) | (nM) | (nM) | (nM) | (nM) |
| 5-HT1A | — | — | — | — | 1284 | 10,000 | 118 | 700 | 673 |
| 5-HT1B | — | — | — | — | — | 10,000 | 10,000 | — | 1837 |
| 5-HT1D | — | — | — | — | — | 10,000 | 106 | — | 1264 |
| 5-HT1E | — | — | — | 89.1 | — | — | 10,000 | — | 10,000 |
| 5-HT1F | — | — | — | 31 | — | — | — | — | — |
| 5-HT2A | 10,000 | 10,000 | 10,000 | 0.4 | 1927 | 239 | 35.8 | 95 | — |
| 5-HT2B | 612 | 258 | 772 | 2.2 | 3.6 | 25 | 189 | 128 | 4134 |
| 5-HT2C | 10,000 | 10,000 | 10,000 | 4.6 | — | 197 | 223.9 | 55.5 | — |
| 5-HT5 | — | — | — | — | — | — | 10,000 | 3710 | 10,000 |
| 5-HT6 | — | — | — | — | — | 10,000 | 10,000 | 1980 | 9080 |
| 5-HT7 | — | — | — | — | 155 | 10,000 | 1782 | 636 | 7306 |
| 5-HT Transporter | — | — | — | — | 2698 | 10,000 | 3616.7 | 990 | 667 |
| Adrenergic Alpha1A | — | — | — | — | — | — | 153 | 10,000 | 269 |
| Adrenergic Alpha1B | — | — | — | — | — | — | — | 10,000 | 142 |
| Adrenergic Alpha2A | — | — | — | — | — | — | 728 | 10,000 | 531 |
| Adrenergic Alpha2B | — | — | — | — | — | — | — | 10,000 | 247 |
| Adrenergic Alpha2C | — | — | — | — | — | — | 155 | 10,000 | 252 |
| Adrenergic Beta1 | — | — | — | — | — | — | 10,000 | 10,000 | 991 |
| Adrenergic Beta2 | — | — | — | — | — | — | 10,000 | 10,000 | 7741 |
| DOPAMINE D1 | — | — | — | — | — | — | 3730 | 10,000 | 10,000 |
| DOPAMINE D2 | — | — | — | — | — | — | 4142 | 10,000 | 10,000 |
| DOPAMINE D4 | — | — | — | — | — | — | 703 | 10,000 | 10,000 |
| DOPAMINE D5 | — | — | — | — | — | | 10,000 | 10,000 | 10,000 |
| Dopamine | | | | | | | | | |
| Transporter | — | — | — | — | — | — | 10,000 | 10,000 | 10,000 |
| Histamine H1 | 153 | 16 | 161 | — | — | 1.3 | 220 | — | 10,000 |
| Histamine H2 | — | — | — | — | — | — | 3290 | 10,000 | 10,000 |
| Histamine H3 | — | — | — | — | — | 402.2 | 10,000 | — | — |
| Norepinephrine | — | — | — | — | — | 10,000 | 10,000 | 1400 | 10,000 |
| Transporter | | | | | | | | | |

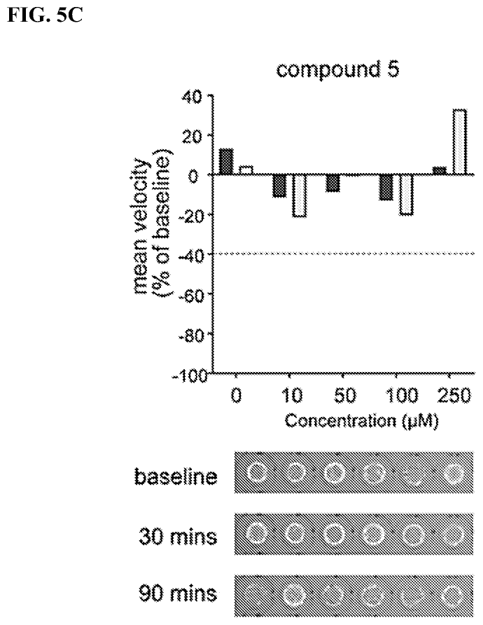

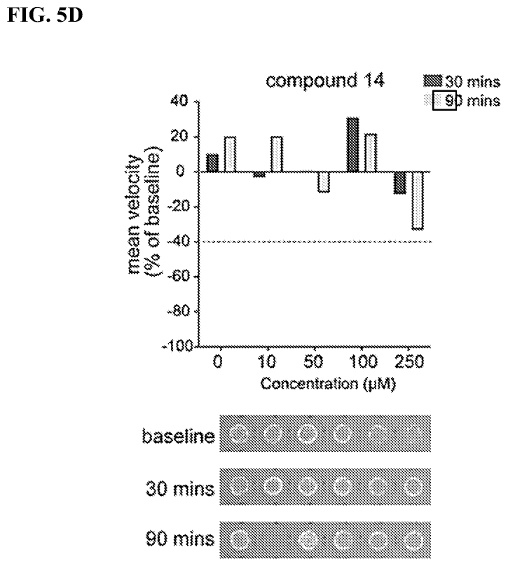

Clemizole analogs with anti-seizure activity selectively bind 5-HT_{2B}R. 5-HT₂R binding affinities for 21 of the clemizole analogs were determined using radioligand binding assays performed by the NIMH Psychoactive Drug Screening Program (Besnard, J. et al., Nature, 492, 215-220, 2012). The three compounds, which exhibited anti-seizure effect on convulsive behaviors of 5 dpf scn1lab mutants, 4, 6 and 20, had significant preference for 5-HT_{2B}R with Ki values of 612 nM, 285 nM and 772 nM, respectively (FIG. 2G-I; Table 2). Additionally, these clemizole analogs showed no significant binding to 5-HT_{2A}R or 5-HT_{2C}R (Ki>10,000 nM). Compounds 5, 14, and 23 also show selectivity for 5-HT_{2B}R with Ki values of 219 nM, 606 nM, and 515 nM. In the initial library screen compounds 5 and 14 had no significant effect on swim behavior and compound 23 was identified as toxic. Additional testing of independently synthesized compound confirmed compounds 5 and 14 have no significant effect on the swim behavior of the scn1lab zebrafish (FIG. 5). Four of the 21 clemizole analogs (compound 10, 15, 17, and 21) showed no significant binding to any human 5-HT₂R.

TABLE 2
Clemizole analogs binding affinity (Ki)
to human 5-HT₂ receptors and H1 receptor

| | 5-HT_{2A}R | 5-HT_{2B}R | 5-HT_{2C}R | H1 |
|---|---|---|---|---|
| Compound | (nM) | (nM) | (nM) | (nM) |
| 1 | >10,000 | 501.0 | 2727.0 | — |
| 2 | — | — | — | — |
| 3 | 327.0 | 83.0 | 292.0 | 4.8 |
| 4 | >10,000 | 612.0 | >10,000 | 153.0 |
| 5 | >10,000 | 219.0 | >10,000 | 362.0 |
| 6 | >10,000 | 285.0 | >10,000 | 16.0 |
| 7 | 15.0 | 46.0 | 345.0 | 1.0 |
| 8 | — | — | — | — |
| 9 | >10,000 | >10,000 | 447.0 | 29.0 |
| 10 | >10,000 | >10,000 | >10,000 | 351.0 |
| 11 | 631.0 | 134.0 | 248.0 | 6.4 |
| 12 | — | — | — | — |
| 13 | — | — | — | — |
| 14 | >10,000 | 606.0 | >10,000 | 27.0 |
| 15 | >10,000 | >10,000 | >10,000 | 865.0 |
| 16 | — | — | — | — |
| 17 | >10,000 | >10,000 | >10,000 | 137.0 |
| 18 | — | — | — | — |
| 19 | — | — | — | — |
| 20 | >10,000 | 772.0 | >10,000 | 161.0 |
| 21 | >10,000 | >10,000 | >10,000 | >10,000 |
| 22 | 623.0 | 306.0 | 4578.0 | 8.5 |
| 23 | >10,000 | 515.0 | >10,000 | 34.0 |
| 24 | 1,259.0 | 115.0 | 1,408.0 | 3.7 |
| 25 | 2,379.0 | 482.0 | 2,265.0 | 76.0 |
| 26 | 316.0 | 20.0 | 313.0 | 1.2 |
| 27 | >10,000 | >10,000 | 7,153.0 | 442.0 |
| 28 | >10,000 | 464.0 | 1084.0 | 44.0 |

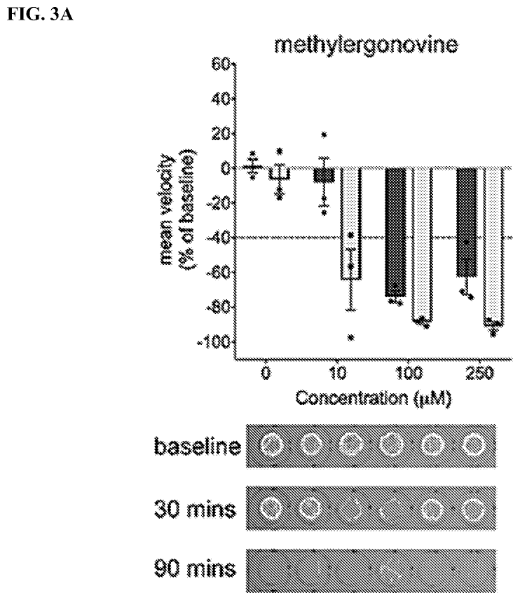

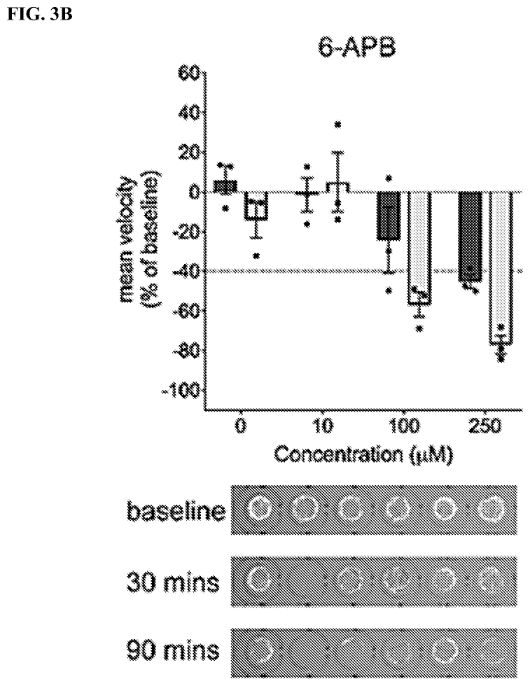

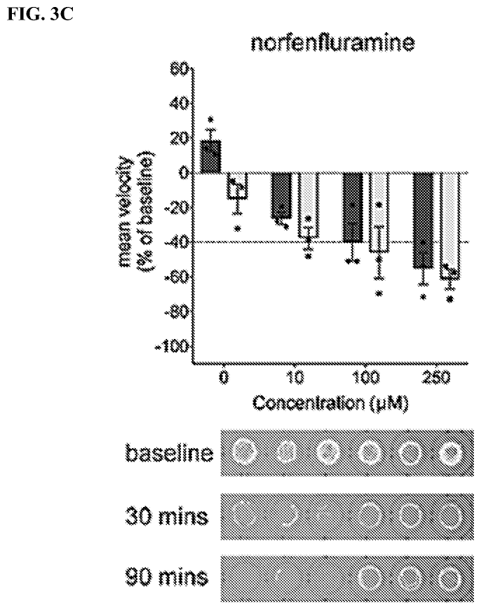

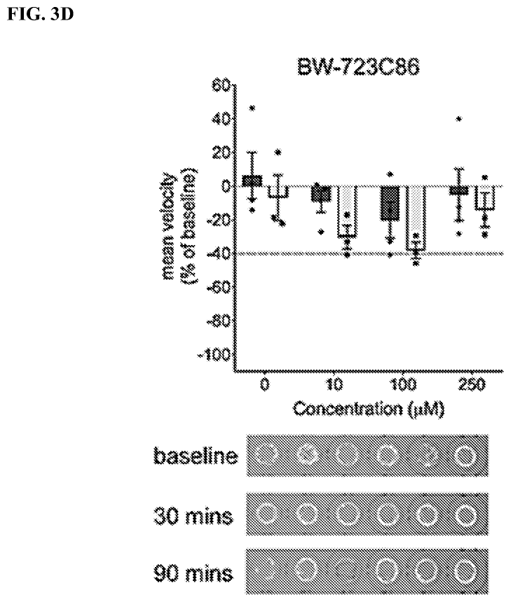

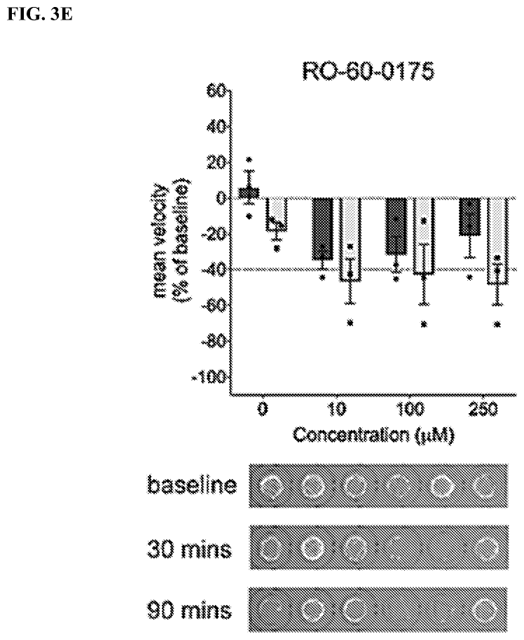

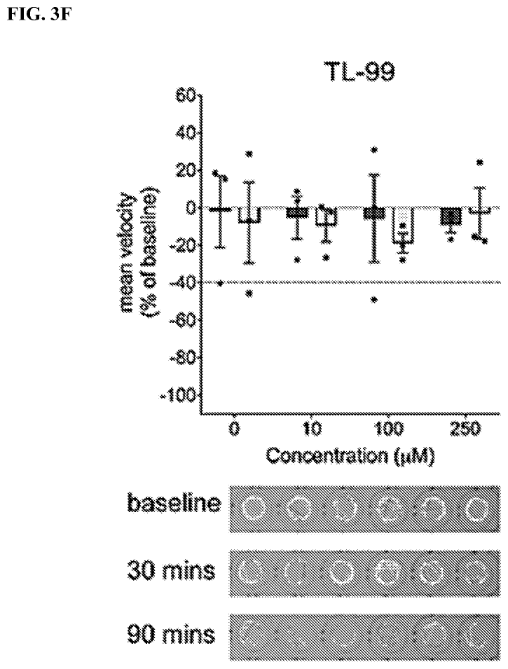

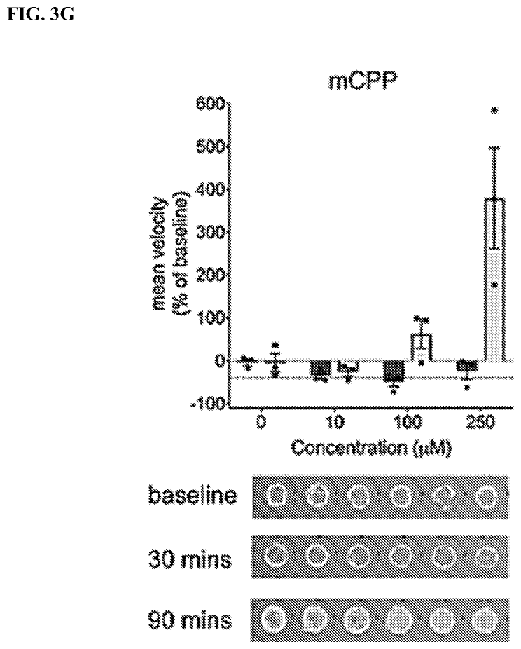

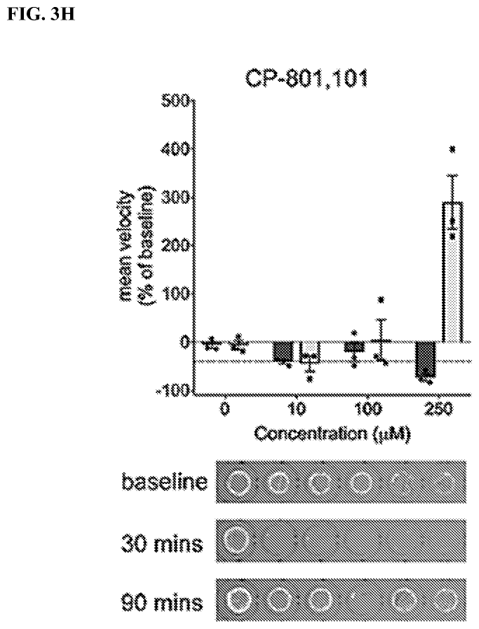

5-HT_{2B}R agonists suppress seizure activity in the scn1lab zebrafish model. A series of commercially available compounds, known to bind 5-HT_{2B}R (Roth, B. L. et al., The Neuroscientist, 6, 252-262, 2000), for their ability to reduce the seizure-like swim behavior of the scn1lab zebrafish larvae (Table 3). Three 5-HT_{2B}R agonists, methylergonovine, 6-APB, and norfenfluramine suppressed convulsive swim behaviors in a concentration-dependent manner (FIG. 3A-C). Additionally, scn1lab mutant larvae treated with 5-HT_{2B}R agonist BW-723C₈₆ (FIG. 3D), showed a decrease in seizure-like swim behavior, but also failed to reach our significance threshold to warrant further testing. Similarly, the 5-HT_{2B}R/5-HT_{2C}R agonist Ro60-0175 consistently decreased mean velocity and was borderline effective after 90 mins of drug exposure (FIG. 3E). Treatment with CP-809,101 gave a biphasic response as did m-CPP, the active metabolite of trazodone (FIG. 3H and FIG. 3G, respectively). TL-99, which had the lowest affinity for 5-HT₂Rs, failed to elicit any behavioral effect in scn1lab mutant larvae (FIG. 3F).

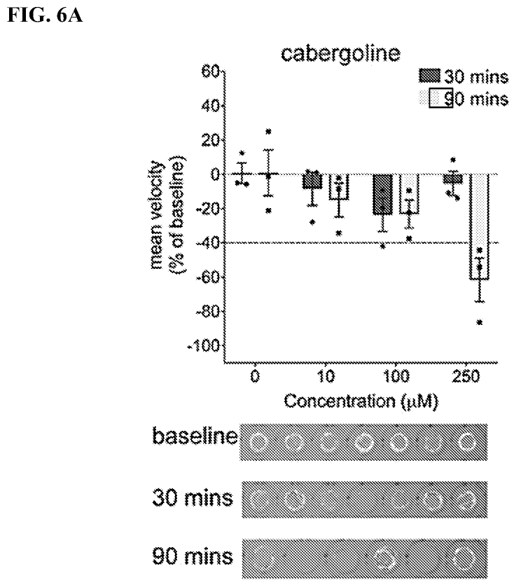

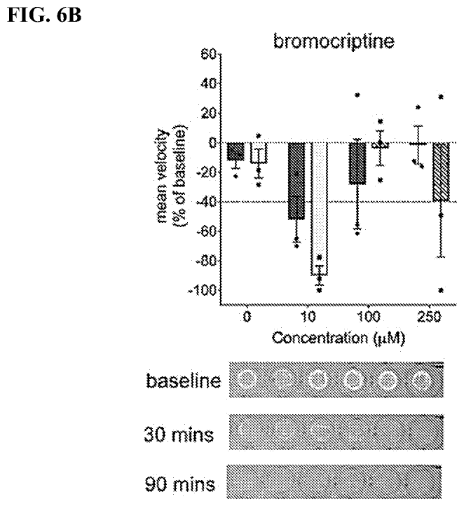

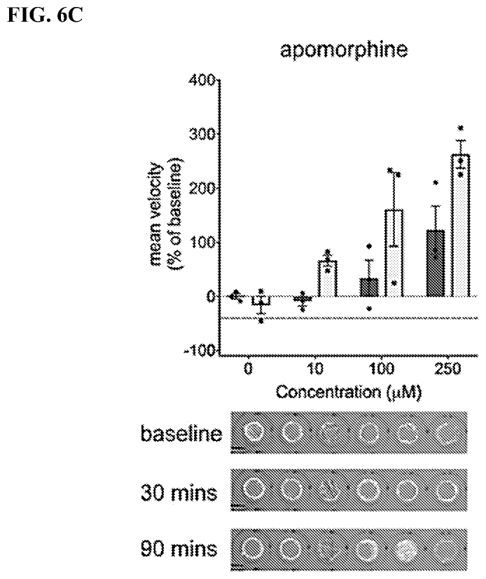

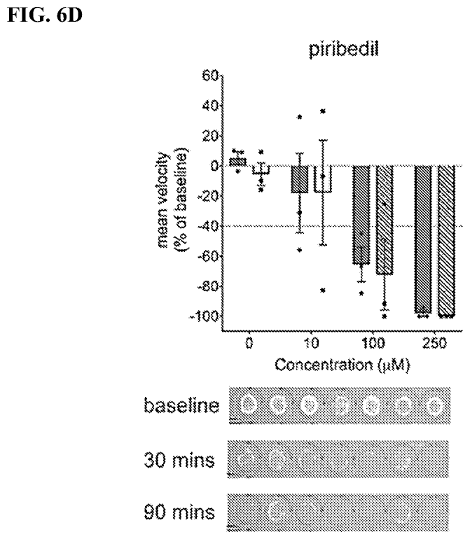

Dopamine receptor agonists with reported 5-HT₂R were also tested for their ability to reduce seizure-like swim behavior (FIG. 6A-D). Cabergoline, a dopamine agonist with recognized high affinity for activating 5-HT_{2B}R (Ki=1.2 nM) significantly reduced convulsive swim-behavior at 250 μM (FIG. 6A), however, due to the lack of a concentration-response it did not undergo further testing. Bromocriptine significantly reduced seizure-like swim behavior at 10 μM (FIG. 6B), however, toxicity was observed at higher concentrations. Piribedil, a dopamine 2 receptor agonist (Ki=1.3 nM), also showed toxicity at 100 and 250 μM (FIG. 6D) and the non-selective dopamine agonist, apomorphine, significantly increased mean swim velocity of scn1lab mutant larvae (FIG. 6C).

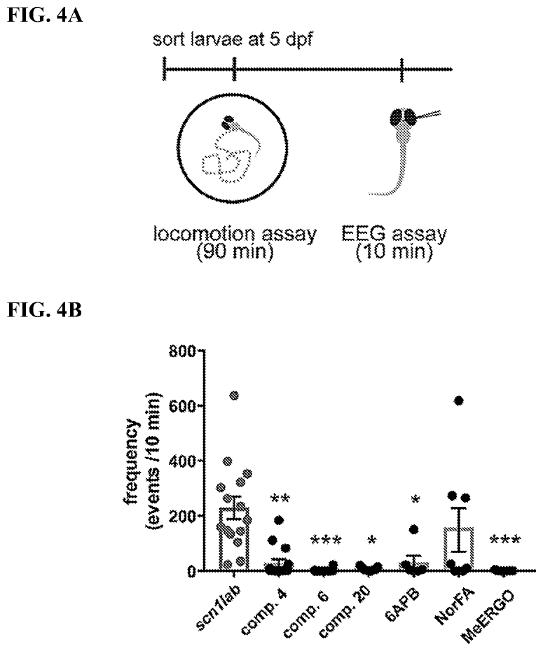

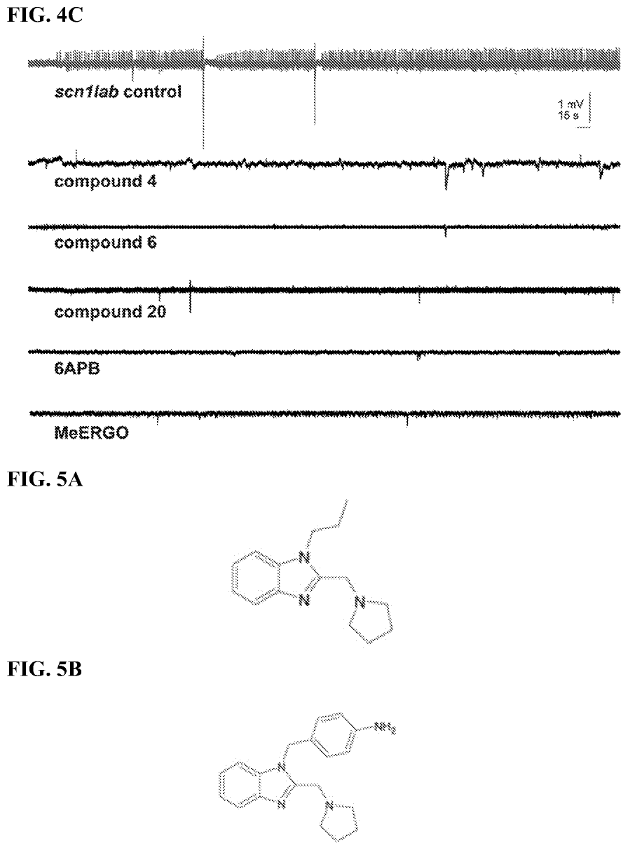

Monitoring electrographic brain activity to confirm seizure suppression is an essential assay to eliminate false positives from behavioral testing (Griffin, A. et al., Frontiers in Pharmacology, 9, 2018). By placing a microelectrode into a visually identified brain region of an agar-immobilized zebrafish larval, stable local field potential (LFP) recordings can be monitored for several hours (Baraban, S. C. et al., Nat Commun, 4, 2410, 2013). At 5 dpf LFP recordings of scn1Lab zebrafish larvae show an average of 250 abnormal electrographic seizure events during a 10 min recording epoch. LFP recordings of scn1lab mutants confirmed significant suppression of electrographic seizure activity after exposure to clemizole analogs 4, 6 and 20 at 100 μM. Representative LFP recording epochs with only the occasional abnormal electrographic event are shown in FIG. 4B and FIG. 4C. Similarly, 250 μM methylergonovine (n=7; p<0.001) or 250 μM 6-APB (n=6; p=0.0238) significantly suppressed the frequency of electrographic seizure events in a manner similar to the 5-HT_{2B}R selective clemizole analogs 4, 6 and 20 (FIG. 4B and FIG. 4C). Radioligand binding data for methylergonovine, 6-APB, and the positively identified clemizole analogs 4, 6 and 20, suggest that all five compounds share a binding affinity for 5-HT_{2B}R (Table 3).

TABLE 3
5-HT_{2B}R binding compounds tested for antiseizure activity.

| | | Drug | 5-HT_{2A}R | 5-HT_{2B}R | 5-HT_{2C}R |
|---|---|---|---|---|---|
| Compound | Main use | Class | (nM) | (nM) | (nM) |
| methylergonovine | smooth muscle | serotonin | 0.4 | 2.2 | 4.6 |
| | constrictor | | | | |
| BW-723C86 | Research use only | serotonin | 89.7 | 3.2 | 114.8 |
| 6-APB | psychoactive drug | serotonin | 1927.0 | 3.6 | — |
| Ro 60-0175 | Research use only | serotonin | 37.2 | 4.3 | 9.1 |
| CP-809,101 | Research use only | serotonin | 1.6 | 6.0 | 64.0 |
| norfenfluramine | fenfluramine | serotonin | 194.0 | 18.0 | 306.0 |
| | metabolite | | | | |
| mCPP | psychoactive drug/ | serotonin | 54.5 | 30.3 | 13.0 |
| | trazodone metabolite | | | | |
| cabergoline | hyperprolactinemia | dopamine | 6.17 | 1.2 | 691.8 |
| bromocriptine | Parkinson's disease | dopamine/ | 107.1 | 56.2 | 741.3 |
| | | serotonin | | | |
| apomorphine | Parkinson's disease | dopamine | 120.2 | 131.8 | 102.3 |
| piribedil | Parkinson's disease | dopamine | >10,000 | 1,202.3 | >10,000 |
| TL-99 | Research use only | dopamine | 2,344.2 | 2,041.7 | 2,290.9 |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of selecting a compound for use in treating epilepsy, said method comprising:
contacting a test compound with 5-hydroxytryptamine-2B receptor (5-HT_{2B}); and
measuring the 5-HT_{2B} agonistic activity of said test compound.

2. The method of claim 1, further comprising an epileptic animal model.

3. The method of claim 2, further comprising administering said test compound to the epileptic animal model and measuring a behavioral activity in said epileptic animal model.

4. The method of claim 3, wherein said epileptic animal model is a Dravet Syndrome (DS) animal model.

5. The method of claim 4, wherein the DS animal model is a zebrafish (Danio rerio) that is resistant to anti-epilepsy drugs (AEDs).

6. The method of claim 5, wherein the zebrafish (Danio rerio) is an scn1lab mutant or an scn1laa mutant.

7. The method of claim 5, wherein the behavioral activity is a convulsive high-velocity swim behavior.

8. The method of claim 2, further comprising administering said test compound to the epileptic animal model and obtaining an electrophysiological recording of the animal model to detect the presence, intensity, or absence of a spontaneous electrographic seizure in said epileptic animal model.

9. The method of claim 8, wherein said epileptic animal model is the DS animal model.

10. The method of claim 9, wherein the DS animal model is the scn1lab mutant zebrafish.

11. The method of claim 1, wherein said measuring comprises determining 5-HT_{2B} receptor binding activity by the test compound.

12. The method of claim 1, wherein said compound has low binding activity to 5-HT_{2A} receptor or 5-HT_{2C} receptor or does not demonstrate measurable 5-HT_{2A} receptor or 5-HT_{2C} receptor binding activity.

13. The method of claim 1, wherein said compound binds to 5-HT_{2B} receptor with a Kd of less than 100 nM, 10 nM, 1 nM, 500 pM, or 100 pM.

14. The method of claim 1, wherein the agonistic activity of said compound to 5-HT_{2B} receptor is 10, 100, 1000, 10000, or 100000 times greater relative to 5-HT_{2A} receptor agonistic activity of said compound.

15. The method of claim 1, further comprising selecting said test compound as said compound based on at least one of (1) 5-HT_{2B} agonistic activity of said test compound, (2) reduced epileptic behavioral activity in said epileptic animal model after administering said test compound to said epileptic animal model, (3) reduction in convulsive high-velocity swim behavior in said epileptic animal model after administering said test compound to said epileptic animal model, (4) detecting a low intensity or absence of a spontaneous electrographic seizure in said epileptic animal model after administering said test compound to said epileptic animal model, (5) binding of said test compound to 5-HT_{2B} receptor with a Kd of less than 100 nM, 10 nM, 1 nM, 500 pM, or 100 pM, or (6) agonistic activity of said test compound that is 10, 100, 1000, 10000, or 100000 times greater relative to 5-HT_{2A} receptor agonistic activity of said compound.

16. A method of selecting a compound for treating an epilepsy, said method comprising:
contacting a test compound with 5-HT_{2B} receptor;
measuring the 5-HT_{2B} agonistic activity of said test compound;
administering said test compound to an epileptic animal model; and
measuring a behavioral activity in said epileptic animal model.

17. The method of claim 16, wherein the epileptic animal model is a scn1lab mutant zebrafish and the agonistic activity of said test compound is measured by one or more of:
convulsive high-velocity swim behavior in the scn1lab mutant zebrafish;
spontaneous electrographic seizures in the scn1lab mutant zebrafish; or
5-HT_{2B} binding activity of said test compound.

18. A method of treating an epilepsy in a subject in need thereof, said method comprising administering to said subject an effective amount of a 5-HT_{2B} specific receptor agonist.

19. The method of claim 18, wherein the agonistic activity of said 5-HT_{2B} specific receptor agonist is 10, 100, 1000, 10000, or 100000 times greater relative to 5-HT_{2A} receptor agonistic activity of said 5-HT_{2B} specific receptor agonist.

20.-23. (canceled)

24. The method of claim 18, wherein said 5-HT_{2B} specific receptor agonist binds to 5-HT_{2B} receptor with a Kd of less than 100 nM, 10 nM, 1 nM, 500 pM, or 100 pM.

25.-28. (canceled)
